SEQUENCE LISTING (30 sequences)

SEQ ID NO: 1 - DNA, 4368 bp, homo sapiens
  CDS (216)..(2183)
atcattgtaaacaggcagaggctgggcggggtgggaatggggcgcccgaggccggcctggggcgcagcgcaggaggcggctccggtggctgcggcggcagcgtgagcgcgaggaggcggaggctgcggcggggcggacggtcgcgcggcggcaggcacaggtgtaatggataggtaacagagaagacctcgtcccttcctagtcagggcatcagcatgactgagtgcttcctgccccccaccagcagccccagtgaacaccgcagggtggagcatggcagcgggcttacccggacccccagctctgaagagatcagccctactaagtttcctggattgtaccgcactggcgagccctcacctccccatgacatcctccatgagcctcctgatgtagtgtctgatgatgagaaagatcatgggaagaaaaaagggaaatttaagaaaaaggaaaagaggactgaaggctatgcagcctttcaggaagatagctctggagatgaggcagaaagtccttctaaaatgaagaggtccaagggaatccatgttttcaagaagcccagcttttctaaaaagaaggaaaaggattttaaaataaaagagaaacccaaagaagaaaagcataaagaagaaaagcacaaagaagaaaaacataaagagaagaagtcaaaagacttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattacgtagagaagtatggcatgaagtgtgaaggcatctacagagtatcaggaattaaatcaaaggtggatgagctaaaagcagcctatgaccgggaggagtctacaaacttggaagactatgagcctaacactgtagccagtttgctgaagcagtatttgcgagaccttccagagaatttgcttaccaaagagcttatgcccagatttgaagaggcttgtgggaggaccacggagactgagaaagtgcaggaattccagcgtttactcaaagaactgccagaatgtaactatcttctgatttcttggctcattgtgcacatggaccatgtcattgcaaaggaactggaaacaaaaatgaatatacagaacatttctatagtgctcagcccaactgtgcagatcagcaatcgagtcctgtatgtgtttttcacacatgtgcaagaactctttggaaatgtggtactaaagcaagtgatgaaacctctgcgatggtctaacatggccacgatgcccacgctgccagagacccaggcgggcatcaaggaggagatcaggagacaggagtttcttttgaattgtttacatcgagatctgcagggtgggataaaggatttgtctaaagaagaaagattatgggaagtacaaagaattttgacagccctcaaaagaaaactgagagaagctaaaagacaggagtgtgaaaccaagattgcacaagagatagccagtctttcaaaagaggatgtttccaaagaagagatgaatgaaaatgaagaagttataaatattctccttgctcaggagaatgagatcctgactgaacaggaggagctcctggccatggagcagtttctgcgccggcagattgcctcagaaaaagaagagattgaacgcctcagagctgagattgctgaaattcagagtcgccagcagcacggccgaagtgagactgaggagtactcctccgagagcgagagcgagagtgaggatgaggaggagctgcagatcattctggaagacttacagagacagaacgaagagctggaaataaagaacaatcatttgaatcaagcaattcatgaggagcgcgaggccatcatcgagctgcgcgtgcagctgcggctgctccagatgcagcgagccaaggccgagcagcaggcgcaggaggacgaggagcctgagtggcgcgggggtgccgtccagccgcccagagacggcgtccttgagccaaaagcagctaaagagcagccaaaggcaggcaaggagccggcaaagccatcgcccagcagggataggaaggagacgtccatctgagcagcctgcgtggccgtctggagtccgtgagactgaaaggacccgtgcatcttactgtaacccgggggccaggccggctctctcgctgtacattctgtaaaggtgtcttctcttctcagactcttcctctgtcacacgtctgactccttcacgtcaggctcaggttccatgggaggacgaagcagtggacgcattgtgggctttagggacagatgagttttccagatagtgtcagcttatttgaagattaattttctttgttaacttaaaataactattttaacccttgagtggcttctttttaaaccaaaaaccgtctttctttgcttttttatcacagcagaatcaggatctctttctcattcaaggggggaaccaccccaggtcagcgctgcgcctgctgtggccgccgcgagccacgccctctgggatctctggtaccgtcactcttgcttgtgccttccacaccttctcggtgcagatccctatgggggagctgcctcacgttctctgactggtcagagcagcgcctggtgggtgttccctggcccactctcctctctccttctgcagttctaaaccacagtctataagcccgagtcaccaggacggcctgtctggccacagacaggggctgcctgtggagcctgcccaccggcccccggcagtgcagtccagcggggaggaggctgcccgttcctgccagttcctcactgcggggaccagcaaaggccttctcactgggttggtcaaaggtagtcaccttggcctggtgcatccacagaggatgttgttcaaaccagaaatcttttaaacgactgaccttccttaaaaacagaatgactccgattgcttgcttgggctagaatgtacacgtctccttgcctgaataagccatatatatgctcttaaacaaaagtttgaaattatccatatcatctcagtgaacctactggtggactcccaattgacaagattgagcaatagaaaaaaattcctttcctttgaatgatagctgtgattcaccccaccccattttcttgtttctggtccatccgatgagacggatgctctgatgctctgaggcttctgggaggctgggccctggaggcaacgtgctgcaggcgcactctgtcagagtgaacagcaccgcgagacaggccaggctcgtggctcggaagacaaaccccacacacactcaaggggtcgaaaacaaaccccacacgagggctctcacctccttctcctaggtagtatttattttcagcacctgtttgatgcagtttttaatcctctacctattgcactgttgtgactcgttggccattatttgatttttgtacgaaaaaaagctttgttatagaaatcagcatactatttttttaaatctggagagaagatattctggtgactgaaagtatggtcgggtgtcagatataaatgtgcaaatgccttcttgctgtcctgtcggtctcagtacgttcactttatagctgctggcaatatcgaaggttccttttttgtttgtgtaaactctaatttctatcaaggtgtcatggatttttaaaattagtatttcattacaaatgtctcagcattggttaactaatttttgccaggaccattattgatcaagcaaataaattcaacagccatttgggaaaaagaaaagcttctagtttttttgtacacattctttctgtgaggagattgagtactctgcagctggcgaggagttggttgaggcacttcttcaaggccaagggggaacacagtgttttgtttccagctcactttgtacccctcacctctgcagacacggggagaaccccggacccctggcatgcatgctggcggcggcatgcctcccttccacaagcccatgctgctgcagagggagcctgtgtttgcaaaacccagtggactgggctgggtctgctgtctgagcagctcctggctccggtgggaactgcacacaagtccactggcctggcttggccccaggcattgcaattgacagacatttgcatttcatacggtaaatgaggactcagcacagccaaccataatcagcatgtctgggatagactggtctagaataaaaatgaagtttccattgctttgtttgctttaaaaattccacaattaaaatatctgtcattgaaagcttaaaaaaaaaaaaaaa

SEQ ID NO: 2 - protein, 655 aa, homo sapiens
MTECFLPPTSSPSEHRRVEHGSGLTRTPSSEEISPTKFPGLYRTGEPSPPHDILHEPPDVVSDDEKDHGKKKGKFKKKEKRTEGYAAFQEDSSGDEAESPSKMKRSKGIHVFKKPSFSKKKEKDFKIKEKPKEEKHKEEKHKEEKHKEKKSKDLTAADVVKQWKEKKKKKKPIQEPEVPQIDVPNLKPIFGIPLADAVERTMMYDGIRLPAVFRECIDYVEKYGMKCEGIYRVSGIKSKVDELKAAYDREESTNLEDYEPNTVASLLKQYLRDLPENLLTKELMPRFEEACGRTTETEKVQEFQRLLKELPECNYLLISWLIVHMDHVIAKELETKMNIQNISIVLSPTVQISNRVLYVFFTHVQELFGNVVLKQVMKPLRWSNMATMPTLPETQAGIKEEIRRQEFLLNCLHRDLQGGIKDLSKEERLWEVQRILTALKRKLREAKRQECETKIAQEIASLSKEDVSKEEMNENEEVINILLAQENEILTEQEELLAMEQFLRRQIASEKEEIERLRAEIAEIQSRQQHGRSETEEYSSESESESEDEEELQIILEDLQRQNEELEIKNNHLNQAIHEEREAIIELRVQLRLLQMQRAKAEQQAQEDEEPEWRGGAVQPPRDGVLEPKAAKEQPKAGKEPAKPSPSRDRKETSI

SEQ ID NO: 3 - protein, 66 aa, artificial sequence
  epitope region comprising residues 154-219
LTAADVVKQWKEKKKKKKPIQEPEVPQIDVPNLKPIFGIPLADAVERTMMYDGIRLPAVFRECIDY

SEQ ID NO: 4 - protein, 15 aa, artificial sequence
  epitope region comprising residues 171-185
KPIQEPEVPQIDVPN

SEQ ID NO: 5 - DNA, 26 bp, artificial sequence
  upstream primer sequence for clone of full length RLIP
ggcggatccatgactgagtgcttcct

SEQ ID NO: 6 - DNA, 34 bp, artificial sequence
  downstream primer sequence for clone of full length RLIP
ccgctcgagtagatggacgtctccttcctatccc

SEQ ID NO: 7 - DNA, 30 bp, artificial sequence
  mutagenic upstream primer for deletion mutant 203-219
gtagagaggaccatggtagagaagtatggc

SEQ ID NO: 8 - DNA, 31 bp, artificial sequence
  mutagenic upstream primer for deletion mutant 154-171
gaagaagtcaaaagacaagccaattcaggag

SEQ ID NO: 9 - DNA, 28 bp, artificial sequence
  mutagenic upstream primer for deletion mutant 171-185
gaagaaaaagaaactcaaacccattttt

SEQ ID NO: 10 - DNA, 31 bp, artificial sequence
  mutagenic upstream prime for deletion mutant 154-219
gaagaagtcaaaagacgtagagaagtatggc

SEQ ID NO: 11 - DNA, 31 bp, artificial sequence
  mutagenic upstream primer for deletion mutant 415-448
gaattgtttacatcgacaggagtgtgaaacc

SEQ ID NO: 12 - DNA, 28 bp, artificial sequence
  mutagenic upstream primer for deletion mutant 65-80
gtgtctgatgataggactgaaggctatg

SEQ ID NO: 13 - DNA, 23 bp, artificial sequence
  targeted cDNA corresponding to nucleotides 508-528 of ral A binding protein 1
aagaaaaagccaattcaggagcc

SEQ ID NO: 14 - RNA, 23 nt, artificial sequence
  corresponding sense siRNA strand for targeted cDNA
gaaaaagccaauucaggagccdd

SEQ ID NO: 15 - RNA, 23 nt, artificial sequence
  corresponding antisense siRNA strand for targeted cDNA
ggcuccugaauuggcuuuuucdd

SEQ ID NO: 16 - RNA, 23 nt, Artificial Sequence
  scrambled sense siRNA strand
guaacugcaacgauuucgaugdd

SEQ ID NO: 17 - RNA, 23 nt, artificial sequence
  scrambled antisense siRNA strand
caucgaaaucguugcaguuacdd

SEQ ID NO: 18 - DNA, 21 bp, artificial sequence
  phosphorothioate antisense oligonucleotides
ggctcctgaattggctttttc

SEQ ID NO: 19 - DNA, 22 bp, artificial sequence
  corresponding silencing RNA sequence to the phosphorothioate antisense oligonucleotide
aagaaaagccaattcaggagcc

SEQ ID NO: 20 - DNA, 198 bp, artificial sequence
  epitope nucleotide sequence corresponding to residues 154-219
ttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattac

SEQ ID NO: 21 - DNA, 45 bp, artificial sequence
  epitope nucleotide sequence corresponding to residues 171-185
aagccaattcaggagccagaggtgcctcagattgatgttccaaat

SEQ ID NO: 22 - DNA, 1968 bp, homo sapiens
  CDS (1)..(1965)
atgactgagtgcttcctgccccccaccagcagccccagtgaacaccgcagggtggagcatggcagcgggcttacccggacccccagctctgaagagatcagccctactaagtttcctggattgtaccgcactggcgagccctcacctccccatgacatcctccatgagcctcctgatgtagtgtctgatgatgagaaagatcatgggaagaaaaaagggaaatttaagaaaaaggaaaagaggactgaaggctatgcagcctttcaggaagatagctctggagatgaggcagaaagtccttctaaaatgaagaggtccaagggaatccatgttttcaagaagcccagcttttctaaaaagaaggaaaaggattttaaaataaaagagaaacccaaagaagaaaagcataaagaagaaaagcacaaagaagaaaaacataaagagaagaagtcaaaagacttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattacgtagagaagtatggcatgaagtgtgaaggcatctacagagtatcaggaattaaatcaaaggtggatgagctaaaagcagcctatgaccgggaggagtctacaaacttggaagactatgagcctaacactgtagccagtttgctgaagcagtatttgcgagaccttccagagaatttgcttaccaaagagcttatgcccagatttgaagaggcttgtgggaggaccacggagactgagaaagtgcaggaattccagcgtttactcaaagaactgccagaatgtaactatcttctgatttcttggctcattgtgcacatggaccatgtcattgcaaaggaactggaaacaaaaatgaatatacagaacatttctatagtgctcagcccaactgtgcagatcagcaatcgagtcctgtatgtgtttttcacacatgtgcaagaactctttggaaatgtggtactaaagcaagtgatgaaacctctgcgatggtctaacatggccacgatgcccacgctgccagagacccaggcgggcatcaaggaggagatcaggagacaggagtttcttttgaattgtttacatcgagatctgcagggtgggataaaggatttgtctaaagaagaaagattatgggaagtacaaagaattttgacagccctcaaaagaaaactgagagaagctaaaagacaggagtgtgaaaccaagattgcacaagagatagccagtctttcaaaagaggatgtttccaaagaagagatgaatgaaaatgaagaagttataaatattctccttgctcaggagaatgagatcctgactgaacaggaggagctcctggccatggagcagtttctgcgccggcagattgcctcagaaaaagaagagattgaacgcctcagagctgagattgctgaaattcagagtcgccagcagcacggccgaagtgagactgaggagtactcctccgagagcgagagcgagagtgaggatgaggaggagctgcagatcattctggaagacttacagagacagaacgaagagctggaaataaagaacaatcatttgaatcaagcaattcatgaggagcgcgaggccatcatcgagctgcgcgtgcagctgcggctgctccagatgcagcgagccaaggccgagcagcaggcgcaggaggacgaggagcctgagtggcgcgggggtgccgtccagccgcccagagacggcgtccttgagccaaaagcagctaaagagcagccaaaggcaggcaaggagccggcaaagccatcgcccagcagggataggaaggagacgtccatctga

SEQ ID NO: 23 - protein, 655 aa, homo sapiens; identical to SEQ ID NO: 2

SEQ ID NO: 24 - DNA, 1980 bp, artificial sequence
  POB1 cDNA
atggaggcggcagcggcggcggcggcggcggcagcggcagcggcagcggcgggcgggggctgtggctccgggccgccgccgctgctgctgagcgagggcgagcagcagtgctactccgagctcttcgcgcgctgtgccggcgccgcgggcgggggccccgggtctgggccccccgaggccgccagagtcgcccccggcacggccactgcggccgccggccccgtggctgacctgtttcgggcatcgcagctgcccgccgagacgctgcaccagatcacagaactgtgtggtgcaaagcgggttggttattttggtccaacacagttttacattgccctgaaattaattgctgcagcacaatctggcctcccggtacggatagagagtattaaatgtgaattgcctctgcctcgctttatgatgtcaaagaatgatggtgagatacgatttgggaacccagctgagctgcatggaactaaggttcagattccatatttaactacagaaaaaaattccttcaaaagaatggacgatgaggataaacaggaaacacagtctcccacgatgtcacccctcgcctcccctccttcttccccgcctcattaccagagggtgcccttgagccatggctacagcaaactgcggagcagcgcagaacagatgcatccagcaccttatgaagctaggcagccccttgtccagcccgagggatcctcatcagggggcccaggaaccaagccccttcggcatcaggcttcccttatccggtccttttcagtggagagggaactacaggataacagcagttaccccgacgaaccctggaggataacagaagaacagcgcgagtactatgtcaatcagttccgatcccttcagccagacccaagctctttcatttcaggttctgtggccaagaacttcttcaccaaatcaaagctttccattccagaactctcctatatatgggagcttagtgatgctgactgtgatggagccctgaccctgcctgagttctgtgctgcgtttcatctcattgtggctcggaagaacggctacccattgcctgagggcctccctccaactctgcagccagaatacctgcaggcagcttttcctaagcccaaatgggactgtcaattatttgattcttattctgagtcactgccggcaaatcaacaacctcgtgacttgaatcggatggagaagacatctgttaaagacatggctgaccttcctgtccctaaccaggatgtaactagtgatgacaaacaagctttgaaaagtactatcaatgaagccttaccaaaggacgtgtctgaggatccagcaactcccaaggattccaacagtctcaaagcaagaccaagatccagatcttactctagcacctccatagaagaggccatgaaaaggggcgaggaccctcccaccccgccacctcggccacagaaaacccattccagagcctcctccttggatctgaataaagtcttccagcccagtgtgccagctaccaagtcaggattgttacccccaccacctgcgctccctccaagaccttgtccatcacagtctgaacaagtgtcggaggccgagttactcccacagctgagcagagccccatcccaggctgcagaaagtagtccagcaaagaaggatgtactgtattctcagccaccatcaaagcccattcgtaggaaattcagaccagaaaaccaagctacagaaaaccaagagccttccactgctgcaagtgggccagcttctgcggcaaccatgaaaccgcatccaacagtccaaaagcagtcttccaaacagaagaaggccattcaaactgctatccgcaaaaataaagaggcaaacgcagtgctggctcggctgaacagtgagctccagcagcagctcaaggaggttcatcaagaacgaattgcattggaaaaccaattggaacaacttcgtccggtcactgtgttgtga

SEQ ID NO: 25 - DNA, 26 bp, artificial sequence
  upstream primer for POB1
ggcggatccatggaggcggcagcggc

SEQ ID NO: 26 - DNA, 29 bp, artificial sequence
  downstream primer for POB1
ccgctcgagtcacaacacagtgaccggac

SEQ ID NO: 27 - protein, 512 aa, artificial sequence
  POB1 deletion mutant 1-512 residues
MEAAAAAAAAAAAAAAAGGGCGSGPPPLLLSEGEQQCYSELFARCAGAAGGGPGSGPPEAARVAPGTATAAAGPVADLFRASQLPAETLHQITELCGAKRVGYFGPTQFYIALKLIAAAQSGLPVRIESIKCELPLPRFMMSKNDGEIRFGNPAELHGTKVQIPYLTTEKNSFKRMDDEDKQETQSPTMSPLASPPSSPPHYQRVPLSHGYSKLRSSAEQMHPAPYEARQPLVQPEGSSSGGPGTKPLRHQASLIRSFSVERELQDNSSYPDEPWRITEEQREYYVNQFRSLQPDPSSFISGSVAKNFFTKSKLSIPELSYIWELSDADCDGALTLPEFCAAFHLIVARKNGYPLPEGLPPTLQPEYLQAAFPKPKWDCQLFDSYSESLPANQQPRDLNRMEKTSVKDMADLPVPNQDVTSDDKQALKSTINEALPKDVSEDPATPKDSNSLKARPRSRSYSSTSIEEAMKRGEDPPTPPPRPQKTHSRASSLDLNKVFQPSVPATKSGLLP

SEQ ID NO: 28 - DNA, 1536 bp, artificial sequence
  POB1 cDNA for deletion mutant 1-512
atggaggcggcagcggcggcggcggcggcggcagcggcagcggcagcggcgggcgggggctgtggctccgggccgccgccgctgctgctgagcgagggcgagcagcagtgctactccgagctcttcgcgcgctgtgccggcgccgcgggcgggggccccgggtctgggccccccgaggccgccagagtcgcccccggcacggccactgcggccgccggccccgtggctgacctgtttcgggcatcgcagctgcccgccgagacgctgcaccagatcacagaactgtgtggtgcaaagcgggttggttattttggtccaacacagttttacattgccctgaaattaattgctgcagcacaatctggcctcccggtacggatagagagtattaaatgtgaattgcctctgcctcgctttatgatgtcaaagaatgatggtgagatacgatttgggaacccagctgagctgcatggaactaaggttcagattccatatttaactacagaaaaaaattccttcaaaagaatggacgatgaggataaacaggaaacacagtctcccacgatgtcacccctcgcctcccctccttcttccccgcctcattaccagagggtgcccttgagccatggctacagcaaactgcggagcagcgcagaacagatgcatccagcaccttatgaagctaggcagccccttgtccagcccgagggatcctcatcagggggcccaggaaccaagccccttcggcatcaggcttcccttatccggtccttttcagtggagagggaactacaggataacagcagttaccccgacgaaccctggaggataacagaagaacagcgcgagtactatgtcaatcagttccgatcccttcagccagacccaagctctttcatttcaggttctgtggccaagaacttcttcaccaaatcaaagctttccattccagaactctcctatatatgggagcttagtgatgctgactgtgatggagccctgaccctgcctgagttctgtgctgcgtttcatctcattgtggctcggaagaacggctacccattgcctgagggcctccctccaactctgcagccagaatacctgcaggcagcttttcctaagcccaaatgggactgtcaattatttgattcttattctgagtcactgccggcaaatcaacaacctcgtgacttgaatcggatggagaagacatctgttaaagacatggctgaccttcctgtccctaaccaggatgtaactagtgatgacaaacaagctttgaaaagtactatcaatgaagccttaccaaaggacgtgtctgaggatccagcaactcccaaggattccaacagtctcaaagcaagaccaagatccagatcttactctagcacctccatagaagaggccatgaaaaggggcgaggaccctcccaccccgccacctcggccacagaaaacccattccagagcctcctccttggatctgaataaagtcttccagcccagtgtgccagctaccaagtcaggattgttaccc

SEQ ID NO: 29 - DNA, 26 bp, artificial sequence
  upstream primer for POB1 deletion mutant 1-512
ggcggatccatggaggcggcagcggc

SEQ ID NO: 30 - DNA, 30 bp, artificial sequence
  downstream primer for POB1 deletion mutant 1-512
ccgctcgagggtaacaatcctgacttggta

(19) United States
(12) Patent Application Publication
(10) Pub. No.: US 2006/0104983 A1
(43) Pub. Date: May 18, 2006

(54) Therapies for the regulation of insulin and glucose using RLIP76

(75) Inventors: Sanjay Awasthi, Arlington, TX (US); Sharad S. Singhal, Arlington, TX (US); Sushma Yadav, Arlington, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(73) Assignee: Board of Regents, The University of Texas System, Austin, TX (US)

(21) Appl. No.: 11/265,065

(22) Filed: Nov. 2, 2005

Related U.S. Application Data
(63) Continuation-in-part of application No. 10/714,506, filed on Nov. 13, 2003.
(63) Continuation-in-part of application No. 10/713,578, filed on Nov. 13, 2003.
(60) Provisional application No. 60/425,917, filed on Nov. 13, 2002.
(60) Provisional application No. 60/425,814, filed on Nov. 13, 2002.

(51) Int. Cl. A61K 48/00; A61K 31/426; C40B 40/08; A61K 39/395

(52) U.S. Cl. 424/155.1; 514/44; 435/6; 514/369

(57) ABSTRACT

The present invention includes methods and compositions used to regulate glucose and insulin levels in subjects in need thereof. Compositions are regions that recognize a ralA binding protein 1 and directly affects transport activity and membrane association of the ralA binding protein 1. The compositions are used to identify chemical compounds (e.g., antibodies, si-RNA and small molecules) that recognize ralA binding protein 1 and to identify medicines used to regulate glucose and insulin levels in subjects in need thereof. Compositions may be used screen chemical libraries for compounds that bind the ralA binding protein 1 and effect its transport activity and/or membrane association.

### CROSS-REFERENCES TO RELATED APPLICATIONS

This application is a continuation-in-part of prior U.S. application Ser. No. 10/714,506, filed Nov. 13, 2003, herein incorporated by reference, which claims the benefit of U.S. Provisional Patent Application No. 60/425,917, filed Nov. 13, 2002. This application is a continuation-in-part of prior U.S. application Ser. No. 10/713,578, filed Nov. 13, 2003, herein incorporated by reference, which claims the benefit of U.S. Provisional Patent Application No. 60/425,814, filed Nov. 13, 2002.

### STATEMENT REGARDING FEDERALLY SPONSORED APPLICATIONS

The U.S. Government has a paid-up license in this invention and the right in limited circumstances to require the patent owner to license others on reasonable terms as provided for by the terms of Grant No. NIH 2-R01-CA77495, Grant No. NIH CA 104661

### REFERENCE TO A SEQUENCE LISTING

This application incorporates by reference sequence listing material included on computer readable form and identified as 124263-1039 RLIP.ST25.txt saved on Nov. 2, 2005, in ASCII readable form.

### BACKGROUND OF THE INVENTION

The present invention relates to improved therapies for glucose and insulin regulation and disorders related to such disruptions, particularly diabetes.

The regulation of blood sugar levels and insulin is critical in humans. When not properly regulated, disorders such as diabetes occur. Present therapies for diabetes are effective but not without problems. Improving on current agents, especially if it prolonged the effectiveness of such agents would provide great benefit to those on daily medication and may lessen dosing of such medications.

### SUMMARY OF THE INVENTION

The present invention solves problems associated with current therapies for glucose- and insulin-related disorders. As provided herein, RLIP76 is found to be membrane associated protein and a critical as well as predominant regulator of medicines that regulate glucose and insulin. Accordingly, chemicals and molecules that directly affect RLIP76 activity and/or its association with the membrane are effective medicines for glucose and insulin regulation for those in need thereof.

In one form, the present invention provides for a critical region of ralA binding protein 1, wherein the region neighbors a membrane-associated portion of the ralA binding protein 1 and directly affects transport activity and membrane association of the ralA binding protein 1. The region is used for screening of chemical compounds to be used as medicinal agents for glucose and insulin regulation for those in need thereof.

Compositions of the present invention include a deletion mutant of POB1 and an internal peptide region of RLIP76 to be used as baits in screens of chemical libraries for synthetic and naturally occurring organic chemicals and compounds with glucose and insulin regulating activity. The identified chemicals and compounds are those acting as specific inhibitors of RLIP76 activity (e.g., found to regulate glucose transport associated with RLIP). As such, the present invention provides for compositions that are improved therapies for glucose and insulin regulation.

In one form, there present invention provides for a region recognizing a ralA binding protein 1, wherein the region further comprises SEQ ID NO.:28 and modified variants thereof. Methods of use of this region are also provided for

In other forms, the present invention provides for compositions and methods of using such compositions, including SEQ ID NO.: 3 to SEQ ID NO.:19, SEQ ID NO.:21 and SEQ ID NO.: 27 to SEQ ID NO.:30. Such compositions, in various forms, to be used to identify compounds capable of glucose and insulin regulation.

Those skilled in the art will further appreciate the above-noted features and advantages of the invention together with other important aspects thereof upon reading the detailed description that follows in conjunction with the drawings.

### BRIEF DESCRIPTION OF THE DRAWINGS

For more complete understanding of the features and advantages of the present invention, reference is now made to the detailed description of the invention along with the accompanying figures, wherein:

FIG. 1 depicts insulin-glucose ratio in RLIP76 deficient and supplemented male mice;

FIG. 2 depicts insulin-glucose ratio in RLIP76 deficient and supplemented female mice;

FIG. 3 depicts RLIP76 protein expression in RLIP76 deficient and supplemented male mice (C=control; R2=RLIP200; R5=RLIP500);

FIG. 4 depicts RLIP76 protein expression in RLIP76 deficient and supplemented female mice (C=control; R2=RLIP200; R5=RLIP500);

FIG. 5 depicts effect of RLIP76 on glucose uptake in H1618 and H358 by comparing wild-type (black bars), control vector transfected (grey bars), or RLIP76 transfected (white bars) H1618 and H358 cells in the absence of insulin;

FIG. 6 depicts the effect of insulin (0.02 IU/ml), anti-RLIP76 IgG, or POB1-liposomes for wild-type (black bars), control vector transfected (grey bars), or RLIP76 transfected (white bars) H1618 cells;

FIG. 7 depicts the effect of insulin (0.02 IU/ml), anti-RLIP76 IgG, or POB1-liposomes for wild-type (black bars), control vector transfected (grey bars), or RLIP76 transfected (white bars) H1618 cells;

FIG. 8 depicts insulin signaling and glucose uptake in HepG2 cells;

FIG. 9 depicts phospho-AKT-1 quantified in in HepG2;

FIG. 10 depicts FOXO-1 DNA-binding activity quantified in HepG2;

FIG. 11 depicts insulin-signaling of Akt-1 in control H1618 cells (black bars) and RLIP76 transfected H1618 cells (white bars);

FIG. 12 insulin-signaling and FOXO-1 DNA-binding activity in H1618 cells;

FIG. 13 depicts depicts insulin-signaling of Akt-1 in control H358 cells (black bars) and RLIP76 transfected H358 cells (white bars);

FIG. 14 depicts insulin-signaling and FOXO-1 DNA-binding activity in control H358 cells (black bars) and RLIP76 transfected H358 cells (white bars);

FIG. 15 depicts a schematic of one manner in which the present invention provides for RLIP-specific inhibitors as improved therapies for regulation of glucose and/or insulin;

FIG. 16 depicts expression and purification of recombinant POB1;

FIG. 17 depicts expression and purification of recombinant POB1¹⁻⁵¹²;

FIG. 18 depicts expression and purification of recombinant RLIP76;

FIG. 19 depicts transport activity of RLIP76 as effected by POB1 (triangles), POB1¹⁻⁵¹² (square) or bovine-serum albumin (diamond) at varying molar ratio using DOX and DNP-SG as transport molecules;

FIG. 20 depicts effect of GSH on ATP-dependent transport of DOX (dark bars) and DNP-SG (light bars) in the presence of excess of POB1;

FIG. 21 depicts effect of increased POB1 on DOX-cytotoxicity using proteoliposomes containing POB1 (triangle), POB1¹⁻⁵¹² (square) or albumin (diamond);

FIG. 22 depicts DOX uptake in H358 cells with control-liposomes (diamond), purified rec-POB1¹⁻⁵¹² liposomes (square), and purified rec-POB1 liposomes (triangle); and

FIG. 23 depicts intact H358 cell efflux studies of DOX with control-liposomes (diamond), purified rec-POB1¹⁻⁵¹² liposomes (square), and purified rec-POB1 liposomes (triangle).

### DETAILED DESCRIPTION OF THE INVENTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

In the description which follows like parts are marked throughout the specification and drawing with the same reference numerals, respectively. The drawing figures are not necessarily to scale and certain features may be shown exaggerated in scale or in a somewhat generalized or schematic form in the interest of clarity and conciseness.

The following are abbreviations that may be used in describing the present invention: RLIP, ral interacting protein; GS-E, glutathione-electrophile conjugates; aa, amino acid.

The causal role of oxidative-stress in mediating insulin-resistance is implied, but the signaling mechanisms responsible remain unknown.

More recently, clathrin-coated pit mediated receptor-ligand pair endocytosis (endocytosis) has received attention because endocytosis is a first step in termination of insulin signaling through internalization of the insulin/insulin-receptor complex. This is regulated by the Ral and Rho-pathways. Ral as well as its down-stream effectors appear to regulate endocytosis of insulin and of growth factor receptors. RLIP76, POB1, and Epsin are among the down-stream proteins in the Ral signaling pathway and appear to form complexes necessary for clathrin-dependent endocytosis of insulin or TGF-β receptors. Ral, along with Ras, is also involved in regulation of phospholipase-D activation that plays an important role in the regulation of endocytosis and is activated in response to insulin-signaling. Ral and phospholipase-D may also regulate vesicle and membrane trafficking.

RLIP76, a Ral-effector and Rho-GTPase-activating protein (GAP), is a transporter of GSH-electrophile conjugate (GS-E) and induced early upon oxidative or radiant stress. These findings directly link Ral-signaling and GSH-homeostasis, and indicate increased RLIP76 activity decreases insulin signaling-time by accelerating endocytosis of receptor/ligand complexes. Along with insulin, RLIP76 is the predominant transporter of GSH-conjugates of lipid-derived electrophilic compounds including lipid alkenals and hydroperoxides as previously determined by the inventors (data not shown). Along with insulin, RLIP76 is increased as an early adaptive response to both heat-shock and oxidative stress as previously determined by the inventors (data not shown).

The present invention illustrates that RLIP76 is directly involved in oxidant-mediated insulin-resistance; increased RLIP76 activity—which would accelerate endocytosis—ounteracts insulin activity and directly confers insulin resistance (which can be modulated by other signaling factors). Accordingly, the present invention provides for RLIP76 as an antagonist of insulin-effects through enhanced endocytosis.

The present invention demonstrates that RLIP76, which is induced during oxidative stress, antagonizes the effect of insulin and links GS-E transport with mechanisms that terminate insulin-signaling through receptor-ligand endocytosis. These novel findings provide an explanation for oxidative-stress mediated insulin-resistance.

The inventors, Sanjay Awasthi and Sharad S. Singhal, of the present invention have recently described a novel non-ABC transporter that appears to be multispecific as a Ral interacting protein (see U.S. application Ser. No. 10/714,506; U.S. application Ser. No. 10/713,578; each incorporated herein by reference). As used herein, this transporter is referred to RLIP76 or RalBP1. The official human genome name for the protein is RALBP1 (SEQ ID NO.:1; and SEQ ID NO.:22 for the coding sequence). RLIP76 is a modular multifunctional and modular protein found ubiquitously in many species from Drosophila to humans. It is encoded in humans on chromosome 18p11.3 by a gene with 11 exons and 9 introns. The protein product, also known as ralA binding protein 1, is typically a 76 kDa (SEQ ID NO.:2; and SEQ ID NO.:23 for the coding sequence) protein; however, splice-variants including a 67 kDa peptide and a longer 80 kDa or 102 kDa peptide, cytocentrin, have also been identified.

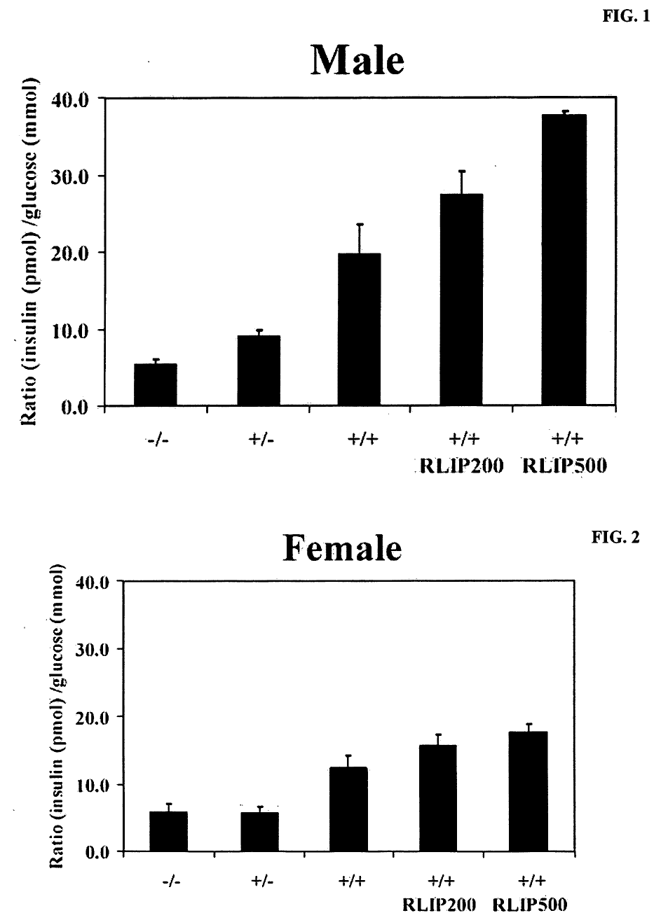

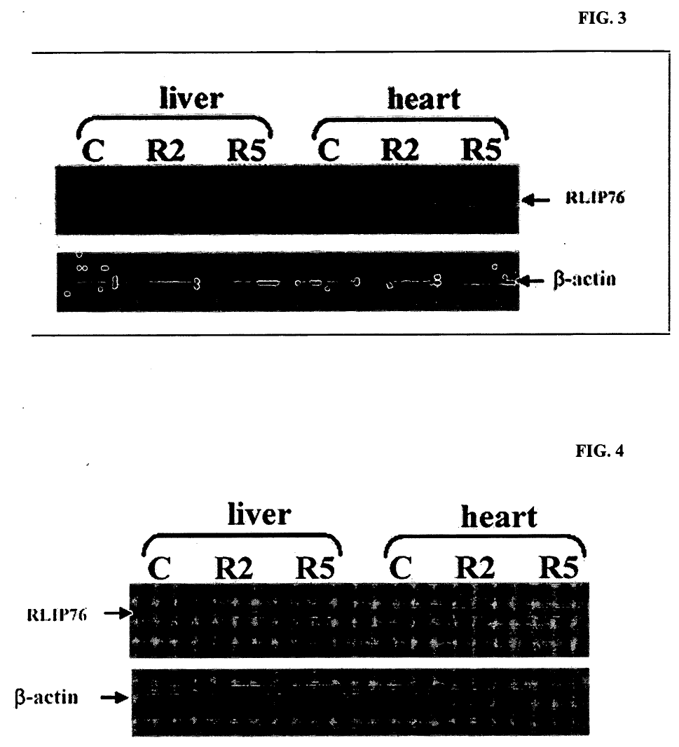

RLIP76 was found to antagonize the effect of insulin on blood glucose in mice. Using knockout mice, colonies were raised and segregated after genotyping from tail tissue. Blood was collected by phlebotomy and subjected to automated analysis for plasma glucose and insulin levels. The results were expressed as ratio of insulin (pmol)/glucose (mmol), which provides an established index for insulin resistance and values from triplicate determinations were presented for the RLIP76^{-/-}, RLIP76^{+/-}, and RLIP76^{+/+} animals, as well as for RLIP76^{+/+} animals treated with intraperitoneal injections of RLIP76-liposomes to augment tissue RLIP76. A progressive increase in insulin/glucose ratio from animals with increasing RLIP76 were found in the following order: RLIP76^{-/-}<RLIP76^{+/-}<RLIP76^{+/+}<RLIP76^{+/+} given 200 μg RLIP76<RLIP76^{+/+} given 500 μg RLIP76 (FIG. 1 and FIG. 2). Higher levels of RLIP76 in the RLIP76^{+/+} mice administered RLIP76 proteoliposomes was confirmed by comparing the results of Western blot analysis from tissues of mice with and without proteoliposomes (FIG. 3 and FIG. 4). Effect of RLIP76 was demonstrable for both genders, though more remarkable in male as compared with the female mice. The greater insulin-sensitivity of RLIP76^{-/-} animals is even more remarkable given that a 2-7 fold increase in lipid-peroxidation products found in the tissue of these animals was not sufficient to mediate insulin-resistance in the absence of RLIP76 (data not shown; disclosed by inventors in Awasthi et al. Cancer Res. 2005; 65:6022-6028). The dose-dependent relationship between RLIP76 levels and insulin-resistance shows that insulin-resistance observed in the presence of oxidative stress is manifested in a dose-dependent manner with respect to ambient RLIP76 concentration/activity.

RLIP76^{+/-} heterozygous knockout animals were commissioned from Lexicon genetics and prepared by a strategy described previously by the inventors in Awasthi et al., 2005 (incorporated herein by reference). Briefly, C57B mice (about 12 weeks old and born of RLIP76^{+/-}×RLIP76^{+/-} mating) were genotyped by polymerase chain reaction strategy. C57B mice that carry heterozygous (+/−) or homozygous (−/−) disruption of the RLIP76 gene were generated and established colonies of RLIP76+/+, RLIP76+/−, and RLIP76−/− C57B mice were prepared by segregation and mating of animals based on genotyping by polymerase chain reaction on tail DNA. Western-blot analysis of various tissues using anti-RLIP76 antibodies confirmed decreased RLIP76 levels in RLIP76+/− mice, and its absence in tissues from RLIP76−/− mice. Consistent with an observed function of RLIP76 as a transporter of GS-E and doxorubicin (DOX) in cell culture studies, GS-E and DOX transport in membrane vesicles obtained from such mice decreased in a stepwise fashion from RLIP76+/+ mice to RLIP76+/− mice to RLIP76−/− mice. Insulin and glucose measurement in blood serum were performed on wild type (RLIP76^{+/+}) animals sacrificed 24 hours after a single intraperatoneal injection of control or RLIP76-liposomes equivalent to 200 and 500 μg RLIP76 protein. Insulin and glucose measurement in blood serum were also performed on heterozygous (RLIP76^{+/-}) and homozygous (RLIP76^{-/-}) animals. The measurements were re-assayed and verified in a separate laboratory. Uptake of RLIP76 by tissues of RLIP76-proteoliposome injected mice was monitored by comparing the results of Western blots of tissues of wild type mice with or without administration of RLIP76 proteoliposomes.

RLIP76 was found to antagonize glucose uptake by cells in culture, thus providing a direct relationship between insulin-mediated glucose uptake and transport activity of RLIP76 in cultured human cell lines. The inventors have previously shown that relatively drug-resistant non-small cell lung cancer cells (NSCLC) have roughly twice the transport activity of RLIP76 and are significantly more resistant to chemotherapy as compared to small cell lung cancer cells (SCLC) (further described in Awasthi, et al., Int. J. Oncol. 2003:22; 713-720). The effect of of RLIP76 on insulin was determined in H1618 and H358 and the same cell types transfected with RLIP76. In addition, the effect of a DNA polymerase alpha-binding proteins, POB1, and anti-RLIP76 IgG was determined (as described in Awasthi, et al., Int. J. Cancer 2003; 106:635-646 and Awasthi, et al. Int. J. Oncol. 2003; 22:713-720). Uptake of ¹⁴C-glucose into cells was determined at 30 minutes after incubation of cells in medium containing radiolabeled glucose, followed by rapid washing to remove extracellular glucose. The effects of insulin, anti-RLIP76 antibody, and purified recombinant POB1-liposomes were also determined by including these in the medium during incubation with ¹⁴C-glucose. Triplicate measurements were performed in two separate experiments, and the effect of each factor was analyzed by factorial ANOVA.

H1618 and H358 cells were transfected with a eukaryotic expression vector alone as control (pcDNA3.1) or with vector containing RLIP76 using a transfection method known to those of ordinary skill (e.g., via Transfection Reagent Kit from Qiagen). Expression of RLIP76 mRNA was evaluated by RT-PCR analysis. RNA was prepared by known methods using RNeasy kit from Qiagen. RNA was quantified and purity was determined by measuring optical density at 260 and 280 run. RLIP76 gene-specific primers (base pairs 334-353 as the upstream primer and base pairs 1209-1228 as the downstream primer) were used for reverse transcription-polymerase chain reaction (RT-PCR). Ready-to-go RT-PCR beads were used according to manufacturer's instructions (Amersham Biosciences). Levels of RLIP76 protein in control and transfected clones were measured by immunoassay using anti-RLIP76 IgG. Aliquots of crude detergent membrane fraction of cells containing 100 μg protein were applied to SDS-PAGE, and Western blot analyses was performed using methods know to one of ordinary skill in the art. The anti-RLIP76 antibody was a polyclonal rabbit-anti-RLIP76 IgG prepared and purified by methods know to one of ordinary skill in the art. For RLIP76 antibodies, recombinant human RLIP76 was expressed in E. coli and purified by DNP-SG-Sepharose affinity purification. Approximately 75 μg was injected into New Zealand White rabbits after obtaining pre-immune serum. After booster doses of 50 μg each at two-week intervals, post-immune serum was obtained. The IgG fraction from pre- and post-immunized heat-inactivated serum was purified by DE-52 anion exchange chromatography, followed by protein-A-Sepharose affinity chromatography. The purity of the antibody was checked by SDS-PAGE as well as Western blotting against goat anti-rabbit IgG. Aliquots of the antibody were stored at −86° C. and checked regularly by aerobic and anaerobic cultures for contamination. Recombinant His-tagged POB1 was purified by Ni-NTA affinity chromatography, and purity was established by SDS-PAGE. The specificity of anti-RLIP and other antibodies were stringently established: purified recombinant RLIP76 used for raising polyclonal antibodies was demonstrated to be homogenous by amino acid composition analysis and showed amino acid yields within 96% of those expected according to its sequence SELDI-MS demonstrating a pattern of [M+H] peaks consistent with homogenous preparations of RLIP76 (data not shown).

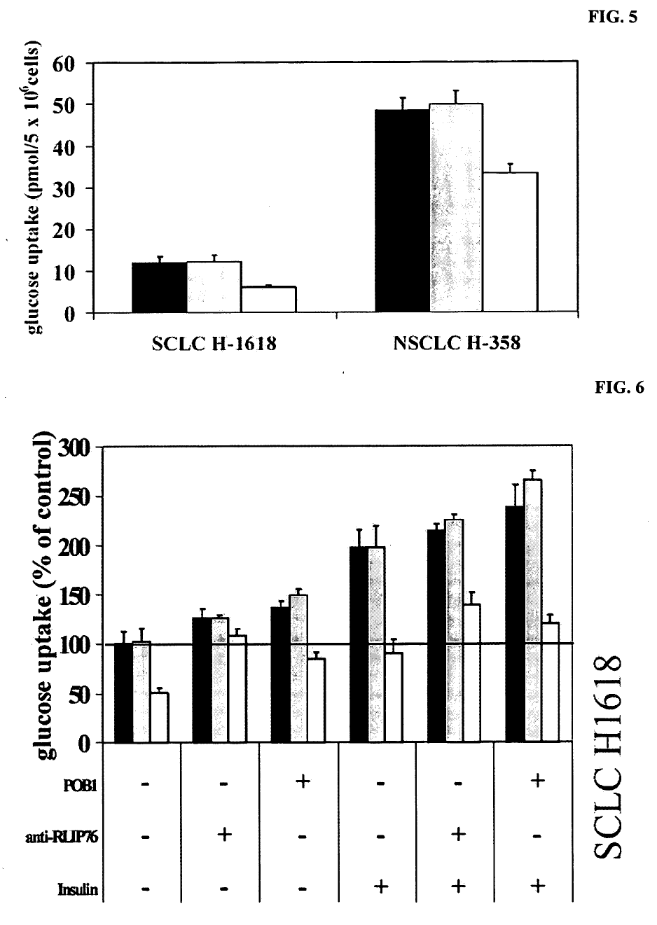

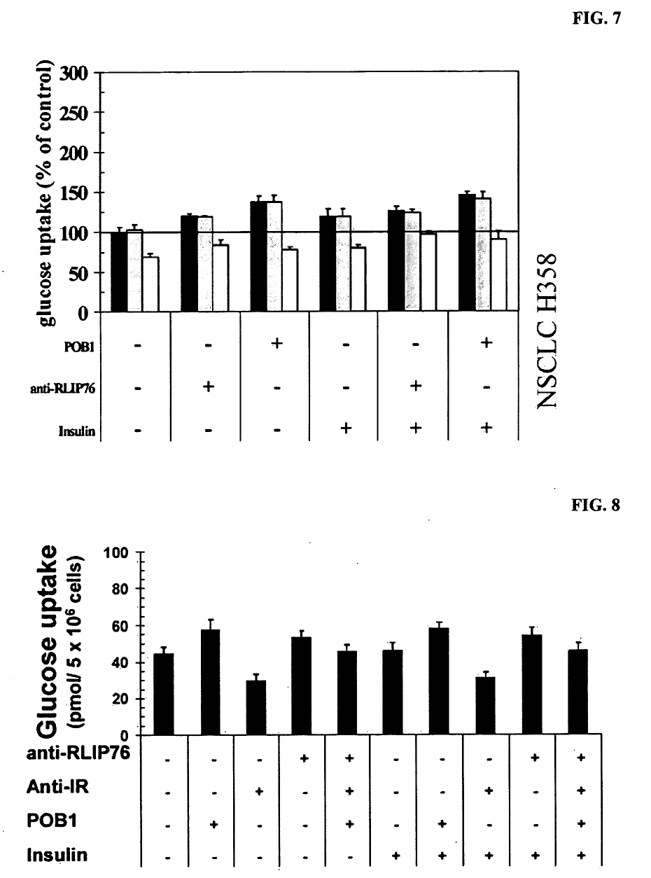

Insulin-independent glucose uptake was several fold-greater in H358 as compared with H1618 cells, thus, transfection of cells with a vector did not effect glucose uptake. RLIP76 transfection reduced glucose uptake significantly in both H358 and H1618 cells (P<0.0001) (FIG. 5). As expected, insulin significantly increased glucose uptake (over 2-fold) in wild-type H1618 (P<0.0001) (FIG. 6). However, in wild-type H358 (FIG. 7), which had about 2-fold higher RLIP76 transport activity as compared to H1618, only a 20% increase in glucose uptake was observed (P<0.05) indicating an inverse relationship between RLIP76 activity and response to insulin. Transfection with empty-vector did not affect insulin-dependent increase in glucose uptake in either cell line, but RLIP76 over-expression resulted in a decreased insulin-responsiveness (P<0.0001).

The inventors have previously demonstrated that anti-RLIP76 antibodies inhibit RLIP76 transport activity in cells and in isolated liposomes or inside out membrane vesicles (as described, e.g., in Awasthi, et al., Int. J. Oncol. 2003:22; 713-720; Awasthi, et al., J. Clin. Invest. 1994; 93:958-965; Awasthi, et al., Int. J. Oncol. 2003; 22:721-732; Awasthi, et al., Biochemistry 2004; 43:16243-16253). Inclusion of anti-RLIP76 antibodies in the media caused an increase in glucose uptake in vector-transfected cells, both in the absence and presence of insulin (P<0.01). Inclusion of POB1 (also previously shown to inhibit transport activity of RLIP76 as described in Yadav, et al., Biochem. Biophys. Res. Commun. 2005; 328:1003-1009) significantly increase insulin-dependent glucose uptake (P<0.0001) demonstrating that inhibition of transport function of RLIP76 augments insulin actions.

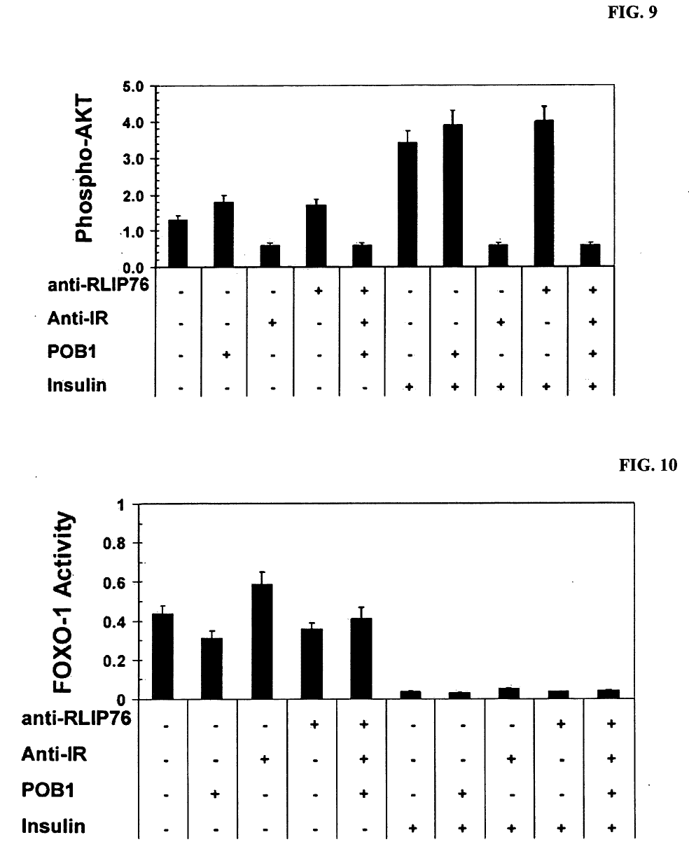

HepG2 cells that lack an ability to stimulate insulin-mediated glucose-uptake did not show a change in glucose uptake in response to insulin (FIG. 8). RLIP76-inhibitors, POB1 and anti-RLIP76 antibodies increased glucose uptake in HepG2 cells whether insulin was included in the medium or whether anti-insulin receptor (anti-IR) were added (FIG. 8). Insulin exposure did increase phosphorylation of Akt-1 and decrease activity of FOXO-1 (Forkhead transcription factor or FKHR) (FIG. 9 and 10). Anti-RLIP76 and POB1 also significantly (P<0.05) increased Akt-1 phosphorylation and significantly decreased FOXO-1 activity. The effect of RLIP76 inhibition on Akt-1 phosphorylation was evident in the presence or absence of insulin, but not apparent with FOXO-1 phosphorylation because at least 90% of FOXO-1 is inhibited by insulin. Anti-IR antibodies not only abrogated insulin effect, but caused the level of phosphorylated Akt-1 to drop below the baseline even in the absence of insulin. Similarly, anti-IR antibody caused a small but significant increase in FOXO-1 activity in the absence of insulin. These confirm that an inhibition of blocking RLIP76 causes an insulin-independent increase in glucose uptake.

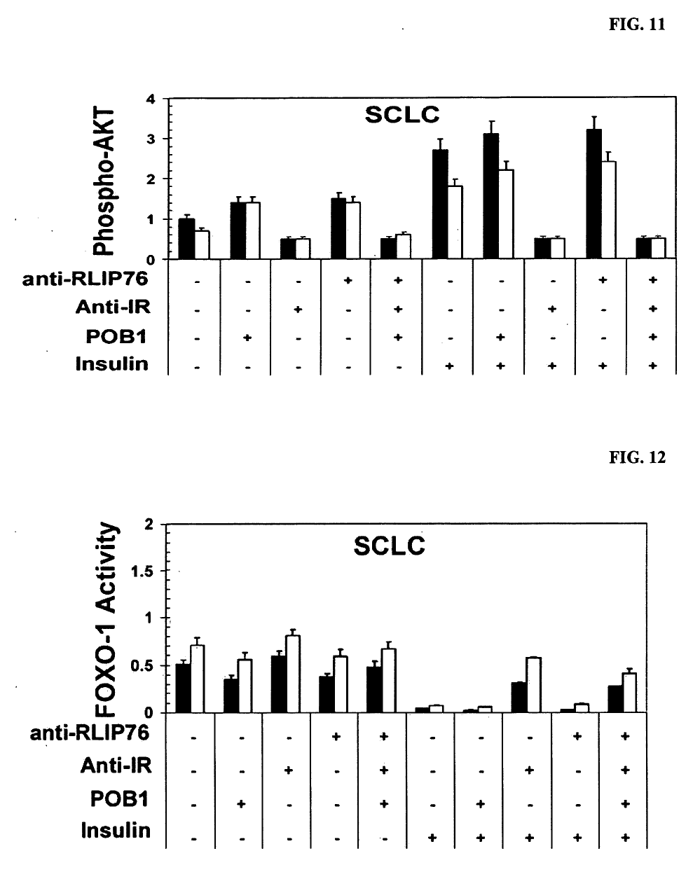

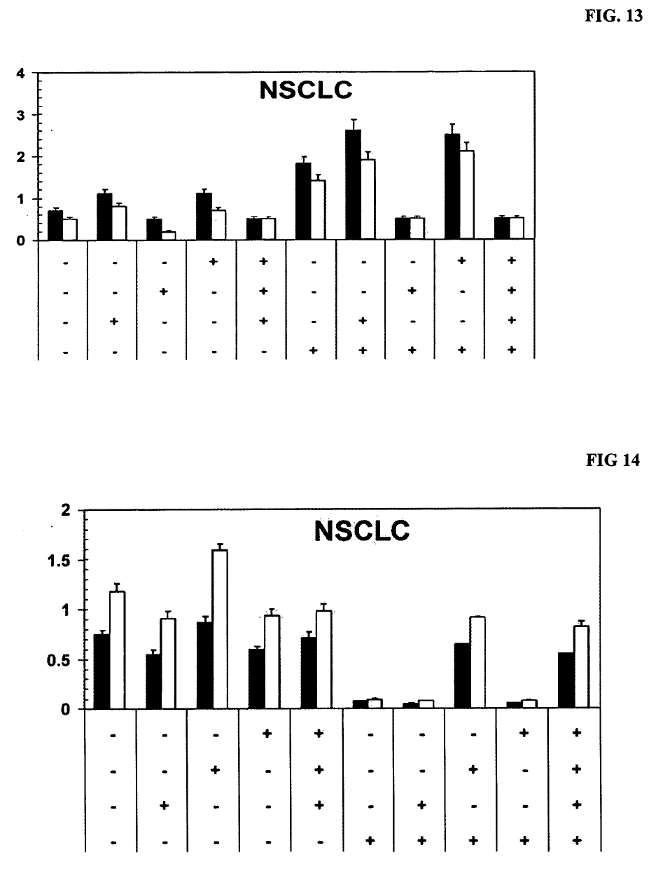

Unlike HepG2, cell lines over-expression RLIP76 did show insulin-mediated increase in glucose uptake. In H1618 cells, over expression of RLIP76 (in the absence of insulin) diminished phosphor-Akt-1, and augmented FOXO-1 activity (FIG. 11 and FIG. 12), effects that are opposite those observed with addition of insulin. Inhibition of RLIP76 by POB1 or anti-RLIP76 IgG antagonized these effects. As in HepG2, insulin caused a marked increase in phospho-Akt-1 and decrease in FOXO-1, both of which were antagonized by RLIP76 over-expression, and augmented by inhibition of RLIP76 using antibodies or POB1. As with HepG2, anti-IR antibody not only abrogated insulin effect, but actually lowered phospho-Akt-1 level (below that observed in the absence of insulin) and increased FOXO-1 activity (above that found in the absence of insulin). In H358 cells, the level of phospho-Akt-1 was also lowered and the level of FOXO-1 raised; levels were greater than those observed in H1618 cells (FIG. 13 and FIG. 14). RLIP76 over-expression caused a significant (P<0.01) decrease in phospho-Akt-1 levels and increased in FOXO-1; effects that are opposite those exerted by insulin. Inhibition of RLIP76 by either POB1 or anti-RLIP76 demonstrated insulin-like effects in the absence of insulin. In the presence of insulin, the effects on phospho-Akt-1 and FOXO-1 were augmented. Thus, RLIP76 antagonizes insulin-action globally, affecting both glucose-uptake and downstream signaling pathways, as expected for RLIP76, functioning as a regulator for endocytosis of the insulin/insulin-receptor complex.

Purified recombinant RLIP76 was reconstituted into artificial liposomes (cholesterol/asolectin) as previously described by the inventors (Awasthi, et al., Biochemistry 2000; 39:9327-9334; Awasthi, et al. Cancer Res. 2005; 65:991-998). Control liposomes were prepared without recombinant protein. The effects of insulin (0.02 IU/mL), anti-RLIP76 IgG vs. pre-immune IgG (40 μg/mL), and POB1-liposome (50 μg protein/mL) vs. control-liposome were analyzed alone and in combinations. Wild type, vector (pcDNA3.1) transfected and RLIP76 transfected H1618 and H358 cells lines were harvested, washed with PBS, and aliquots containing 5×10⁶ cells (in triplicate) were inoculated into Hanks buffer. After 4 hours incubation at 37° C., cells were pelleted and resuspended in a buffered solution containing either anti-RLIP76-IgG or POB1-liposomes, and incubated at 37° C. for 60 minutes prior to addition of buffer without or with insulin. ¹⁴C-glucose (specific activity 93 cpm/pmol) was then added to the medium and incubated for additional 30 minutes at 37° C. Cells were centrifuged in the cold, medium removed and cell pellets washed twice with a cold phosphate-buffered saline. Radioactivity was determined in the cell pellet. Counts were normalized to the no-IgG control for the respective cell line.

A DNA-binding ELISA kit was used for understanding transcription factor activation of FOXO1 in mammalian cell culture extracts. Colorimetric transcription factor measurements were performed according to the manufactures instructions. Briefly, 40 μL of a binding buffer was added to each well used from a 96-well plate in which multiple copies of specific double-stranded oligonucleotides had been immobilized. 10 μL of cell lysate containing 50 μg protein diluted in a lysis buffer was added to each well. For a blank, 10 μL of lysis buffer was used. The plates were sealed with an adhesive cover and incubated for 1 hour at room temperature with mild agitation. Each well was washed three times with a wash buffer followed by addition of 100 μL diluted FKHR primary antibody (1:500 dilution in an antibody binding buffer) to each well. The plate was covered and incubated at room temperature for 1 hour without agitation followed by washing with 200 μL of wash buffer. 100 μL diluted horseradish peroxidase-conjugated secondary antibody (1:1000 dilution with an antibody binding buffer) was added to each well and the plate was incubated at room temperature for 1 hour followed by adding 100 μL developing solution to each well. The plate was incubated 2-10 minutes at room temperature protected from light until the wells turned blue. The reaction was stopped by adding 100 μL of a stop solution and the absorbance was read at 450 nm using an ELISA plate reader.

After treatment with either liposomes or anti-RLIP76 IgG or insulin, all cells (H1618 control and RLIP76 transfected; H358 control and RLIP76 transfected; and HepG2) were pelleted and lysed in a lysis buffer (10 mM Tris-HCl, pH 7.4, containing 1.4 mM β-mercaptoethanol, 1 mM EDTA, 0.1 mM PMSF, 0.05 mM BHT and 0.5% polidocanol) by sonication. Cell lysates containing 50 μg of protein were subjected to SDS-PAGE in 12% gel. Western blot analyses were performed using rabbit antibodies against human anti-phospho-Akt1 (from Upstate Cell Signaling, New York) and peroxidase-conjugated goat anti-rabbit IgG as secondary antibodies. Blots were developed by a chemiluminescence's detection system.

RLIP76 effects endocytosis of insulin-insulin receptor (I-IR) as shown by comparing the uptake of rhodamine-labeled epidermal growth factor (EGF) in wild-type and RLIP76-overexpressing H358 transfected cells. The internalization of EGF was considerably quicker in RLIP76 over-expressing cells; anti-RLIP76 antibodies markedly reduced EGF internalization (data not shown). Substituting rhodamine-tagged insulin for rhodamine-EGF also lead to a marked increase in the rate of endocytosis of rhodamine-insulin with increased RLIP76 expression; the increase was dramatically reduced by addition of anti-RLIP76 IgG. Cells (0.5×10⁶ cells/mL) were grown overnight on sterilized glass cover slips (18 mm size) in tissue culture-treated 12-well plates followed by removal of the growth media. Cells were then treated with 10% goat serum for 30 minutes at room temperature and incubated with pre-immune serum as well as with anti-RLIP76 IgG for 2 hours at room temperature. After washing, cells were incubated with 40 ng/mL EGF-rhodamine for 60 minutes in the cold. Cells were then incubated at 37° C. in a humidified chamber for 1, 5 or 10 minutes followed by fixation with a 4% paraformaldehyde solution. Slides were analyzed using confocal laser scanning microscopy with excitation at 555 nm and emission 580 nm.

Transient oxidative stress causes increased endocytosis of I-IR. The inventors have previously demonstrated a rapid induction of RLIP76 in cells exposed to mild transient oxidative stress along with a transport rate of GS-E that is several-fold faster as protection from toxicity of electrophilic products of lipid peroxidation. H358 cells were subjected to mild oxidative stress for a short time as H202 or 25 mM glucose, both of which lead to a marked increase in endocytosis of the insulin receptor. Anti-RLIP76 antibodies inhibited insulin-endocytosis in these studies as well (data not shown). Thus, RLIP76 plays a role in receptor-ligand endocytosis associated with transient glucose intolerance caused by stress and shows that GS-E transport by RLIP76 is involved in this receptor-ligand pair endocytosis.

H358 cells (0.5×10⁶ cells/ml) were grown on sterilized glass cover slips (18 mm size) in RPMI 1640 medium in tissue culture treated 12 well plates for overnight, followed by incubation with either 50 μM H₂O₂ or 25 mM glucose for 20 min at 37° C. Subsequently, cells were washed and resuspended in the RPMI 1640 medium and allowed to recover for 2 hours at 37° C. CO₂ incubator. Cells were washed and treated with 10% goat serum for 30 minutes at room temperature and incubated with 100 ng/mL FITC-conjugated insulin (prepared in PBS) for 60 minutes on ice. Cells were then incubated at 37° C. in humidified chamber for 1, 5 or 10 minutes followed by fixation with 4% paraformaldehyde in PBS at respective time. Slides were analyzed using confocal laser scanning microscopy with excitation at 494 nm and emission 518 nm.

H358 cells (approximately 50×10⁶) grown in culture flask, were treated with either 50 μM H₂O₂ or 25 mM glucose for 20 minutes at 37° C. followed by washing, resuspending in growth medium and stabilizing for 2 hours at 37° C. Cells were then pelleted, solubilized in a lysis buffer (10 mM Tris-HCl, pH 7.4, 1.4 mM β-mercaptoethanol, 100 μM EDTA, 50 μM BHT, and 100 μM PMSF containing 1% polidocanol), sonicated for 30 seconds and incubated for 4 hours in the cold with gentle shaking. After incubation, each reaction was centrifuged in the cold for 1 hour and supernatants were collected. 100 μg proteins were subjected to SDS-PAGE followed by western blot analyses using anti-RLIP76 IgG.

A proposed model of RLIP76 activity in glucose and insulin regulation is provided herein in which glutathione-conjugate transport functions as a determinant of the rate of clathrin-coated pit mediated receptor-ligand endocytosis. During oxidative stress, an increase in cellular glutathione-conjugates results in increased transport of physiological substrates transport allocrites of RLIP76 (e.g., leukotriene and 4-HNE), which accelerates the rate of receptor-ligand pair endocytosis. This contributes to a relative decrease in the effectiveness of a ligand to initiate intracellular signaling due to a lack of sufficient time for a ligand-receptor interaction. More specifically, increased rate of endocytosis of insulin/insulin-receptor complex during oxidative stress results in relative insulin-resistance. Inhibition or decrease in transport activity of RLIP76 provides an opposite effect of insulin-sensitization.

The proposed model is supported by activity in an animal model (mice) and in an cell models. In the animal studies, the present invention demonstrates that blood glucose and insulin levels are significantly perturbed in RLIP76 knockout animals. The ratio of blood insulin/glucose, a surrogate for insulin-resistance, is remarkably lower in RLIP76^{-/-} animals compared with either the RLIP76^{+/+} or RLIP76^{+/-} animals, indicating increased insulin-sensitivity. A dose-dependent increase in insulin-resistance also supports the model. Also consistent with the model is the decreased effectiveness of insulin-mediated glucose uptake in cultured cells with naturally high levels of RLIP76 and antagonism of this effect through inhibition of RLIP76 (via anti-RLIP76 antibody or POB1).

Indeed the present invention indicates that insulin-resistance associated with clinical disease conditions may often include an increase in oxidative stress. Thus, inhibition of RLIP76 may be an important addition to therapies provided for those having such clinical conditions. The above examples demonstrate the previously unrecognized and unanticipated activity of RLIP76 as a significant transporter involved in the regulation of glucose and insulin. In addition, the activity of RLIP76 is believed to be directly involved in disorders related to the disregulation of glucose and/or insulin.

Potency of RLIP76 appears to be enhanced with glucose and/or insulin disregulation. Therefore, the present invention provides for compositions that antagonize RLIP76 (e.g., inhibit RLIP76 transport activity); such compositions being new and important hypoglycemic agents to be used alone or in combination with existing therapies. The antagonists include compounds that interact with a cell-surface domain of RLIP76 that directly affects RLIP76 transport activity or those that interact with RLIP76 through its POB1 binding site. Suitable compositions include those molecules that inhibit or reduce transport activity of RLIP76 and may be identified as described below.

The inventors have recently reported several surface epitope regions of RLIP76 when membrane bound to cells. The surface epitope region was found necessary for optimal transport activity of RLIP76 as described, e.g., in Yadav, et al., Biochemistry 2004; 43:16243-53, herein incorporated by reference. One surface epitope region comprises on or about amino acids (aa) 154 to 219 (SEQ ID NO.:3 and conservatively modified variants, thereof, including deletions of 1 to 5 residues at the C-terminus and or N-terminus). The corresponding DNA/RNA sequence for this surface epitope region is SEQ ID NO.: 20. Another surface epitope region comprises on or about amino acids 171 to 185, corresponding to an aa sequence KPIQEPEVPQIDVPN (SEQ ID NO:4 and conservatively modified variants, thereof, including deletions of 1 to 5 residues at the C-terminus and or N-terminus, with a corresponding DNA/RNA sequence of SEQ ID NO.:21). Such surface epitope regions are not only necessary for optimal transport activity, they are also useful portions of the protein for the identification of inhibitors of RLIP76 transport activity. For example, a deletion mutant protein lacking amino acids 171 to 185 resulted in loss of hydrophobicity of the protein, decreased association of the protein with artificial liposomes, and decreased transport activity. In addition, cells transfected with 171-185 si-RNA (SEQ ID NO.:5) resulted in loss of cell surface expression (e.g., decreased membrane association).

Accordingly, the present invention identifies regions of the protein acting as surface epitopes and capable of providing antagonists (e.g., inhibitors) for RLIP76. Antagonists, as identified herein, include antibodies directed against one or more surface epitope region, si-RNA sequences directed against one or more surface epitope regions, as well as small molecules found using chemical library screenings against peptides containing one or more surface epitope regions.

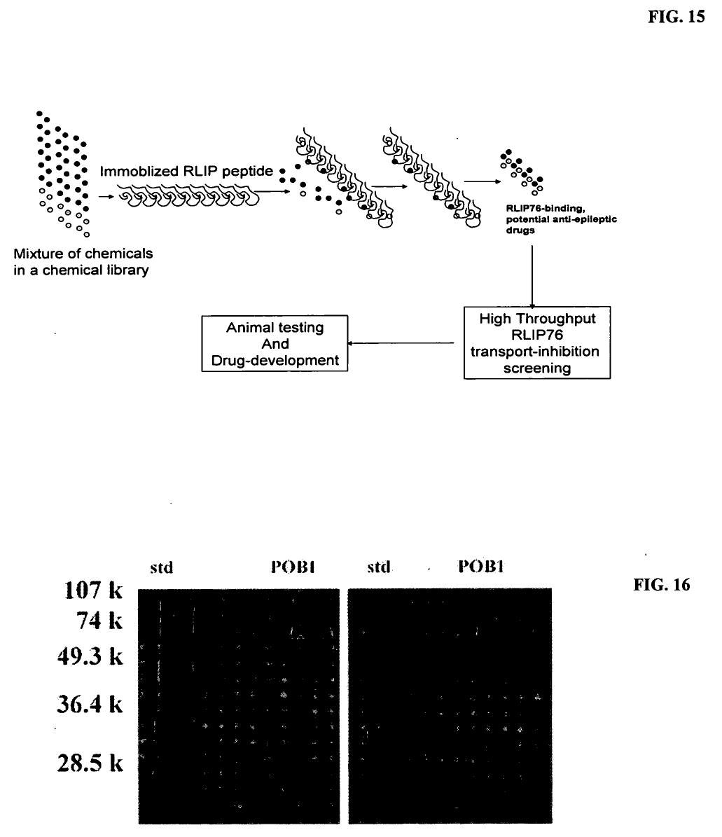

In one form, surface epitope regions and their variants, as identified herein, are synthesized and immobilized on an inert support material and used to screen chemical libraries for compounds that bind this peptide as shown in FIG. 15. Suitable methods for chemical library screening are known to one of ordinary skill in the art. The compounds identified by the screening process are tested in a secondary screen that included a liposomal transport assay to determine efficiency of inhibition of RLIP76. RLIP76 inhibitors are also tested in animals alone and in combination with existing medicines for regulation of insulin and/or glucose in order to evaluate safety and efficacy of each identified inhibitor.

SEQ ID NO:3 and SEQ ID NO:4 were identified from a series of deletion mutant proteins to RLIP76 (data not shown; see Yadav, et al., 2004). In brief, a series of deletion mutants were prepared by PCR-based site-directed mutagenesis using a clone of the full length RLIP76 in an expression vector [pET30a(+)] as template and upstream primer 5′ GGCGGATCCATGACTGAGTGCTTCCT (SEQ ID NO.:5;: BamH1 restriction site is underlined) and downstream primer 5′ CCGCTCGAGTAGATGGACGTCTCCTTCCTATCCC (SEQ ID NO.:6; XhoI restriction site underlined). Mutants included those having deletions of amino acids 203 to 219 (del 203-219), 154 to 171 (del 154-171 ), 171 to 185 (del 171-185), 154 to 219 (del 154-219), 415 to 448 (del 415-448) and 65 to 80 (del 65-80). The mutagenic primers for del 203-219: 5′ GTAGAGAGGACCATGGTAGAGAAGTATGGC 3′ (SEQ ID NO.: 7) with its reverse complement); for del 154-171 : 5′ GAAGAAGTCAAAAGACAAGCCAATTCAGGAG (SEQ ID NO.: 8; with its reverse complement); for del 171-185: 5′ GAAGAAAAAGAAACTCAAACCCATTTT 3′ (SEQ ID NO.: 9; with its reverse complement); for del 154-219: 5′ GAAGAAGTCAAAAGACGTAGAGAAGTATGGC 3′ (SEQ ID NO.: 10; with its reverse complement; for del 415-448: 5′ GAATTGTTTACATCGACAGGAGTGTGAAACC (SEQ ID NO.: 11; with its reverse complement); and for del 65-80: 5′ GTGTCTGATGATAGGACTGAAGGCTATG 3′ (SEQ ID NO.: 12 and its reverse complement).

The template and each deletion mutant was expressed in E. coli and after bacterial lysis (with e.g., 1% (w/v) C₁₂E₉ in lysis buffer), the protein was extracted by methods known to one of ordinary skill in the art. For the full length protein and the deletion mutants, one method of protein purification to nearly homogeneity from bacterial extracts used DNP-SG affinity resins (for full description see Awasthi, et al. Biochemistry 2000:39:9327-9334, herein incorporated by reference). Introduction of deletions specified above in wild type RLIP76 did not affect the affinity of protein with DNP-SG; all deletion mutants could be purified by DNP-SG affinity chromatography. Protein purity was ascertained by SDS-PAGE, Western blot and amino acid composition analysis using methods know in the art and as described in Awasthi et al. 2000. The authenticity of the mutation and the absence of other fortuitous mutations were confirmed by DNA sequencing for each of the deletion mutants.

Full-length RLIP76 (wt-RLIP76) and deletion mutants (del 203-219, del 154-171, del 171-185, del 154-219, del 65-80, del 415-448 and del 65-80) were expressed as recombinant (rec) proteins in E. coli (using pET30a(+) plasmid under the control of the lac UV5 promoter. Single bacterial colonies were used to induce protein expression. To facilitate extraction of the rec-RLIP76 and its various deletion mutants, bacterial lysates were collected, sonicated, and incubated. After incubation, each reaction mixture was centrifuged and the supernatant fraction was obtained as a cytosol fraction and the pellet was the membrane fraction. The membrane fraction was resuspended in 1% polidocanol (a non-ionic detergent) sonicated again, incubated and collected in the supernatant after centrifugation.

When extracted in detergent-containing buffer, the ratio of RLIP76 in the detergent/aqueous extracts was found to be 2.5 for the wild-type protein, but decreased to 0.7 in the mutant in which aa 154-219 (SEQ ID NO.:4) were deleted (data not shown; see Yadav, et al., 2004). Deletion of only one segment of this region (del 171-185 or SEQ ID NO.:3) alone resulted in a significant decrease in this ratio to 1.0. For the mutants with deletions within the region from aa 154-219, loss of hydrophobicity correlated with decreased incorporation of mutants into artificial liposomes, and decreased transport activity. The data indicates that the 154-219 region of RLIP76 significantly affects protein partitioning between cytosol and membranes; residues 171-185 contribute significantly to this effect.

Functional reconstitution of purified RLIP76 from E. coli for transport studies was performed using methods known to one of ordinary skill in the art, an example of which is described in Awasthi, et al. 2000. The degree of incorporation of wild-type as well as mutant RLIP76 into artificial liposomes was assessed by measuring RLIP76 after centrifugation (pellet and supernatant of prepared liposomes) by ELISA assay using anti-RLIP76 antibodies. Measurement of the transport of a cationic agent, doxorubicin (DOX), in the reconstituted liposomes was performed using methods known to one of ordinary skill in the art, an example of which is described in Awasthi, et al. 2000. The ATP-dependent uptake of [¹⁴C]-DOX (specific activity 8.4×10⁴ cpm/nmol) was determined by subtracting the radioactivity (cpm) of the control without ATP from that of the experimental containing ATP. Transport of DOX was calculated in terms of pmol/min/mg protein. The transport of [³H]-DNP-SG (specific activity 3.2×10³ cpm/nmol) was measured using methods known to one of ordinary skill in the art, an example of which is described in Awasthi, et al. 2000.

The majority (87%) of total wild-type RLIP76 was found in the pellet fraction, incorporated into the proteoliposomes. Deletion of aa 203-219 or 154-171 decreased incorporation slightly (to 83 and 80%, respectively). Deletion of aa 171-185 significantly effected incorporation of the protein into proteoliposomes (64%) as did deletion of residues 154-219, with only 33% of total protein found incorporated into proteoliposomes. Deletions affecting the ATP-binding sites (aa 65-80 and aa 415-448) had no significant effect on the amount of protein incorporated into proteoliposomes. Thus region 154-219 is an important determinant of membrane insertion.

For ATP-dependent transport of molecules across the proteoliposomes, transport was significantly decreased (21%) in the mutant lacking aa 154-171 (27.6 nmol/min/mg versus 21.7 nmol/min/mg for transport of DOX by the full length RLIP76 versus the deletion mutant, P<0.05). Deletion of aa 171-185 resulted in approximately 40% loss of transport activity for DOX and a similar loss (35%) in transport activity for dinitrophenyl S-glutathione (DNP-SG). Deletion of the entire 154-219 region resulted in further significant loss (50%) of transport activity for both DOX and DNP-SG. Because deletion of ATP-binding site regions did not affect partitioning of the mutants between cytosol and membrane, the observed decrease in transport activity of deletion mutant aa 154-219 is believed due to loss of protein association with the membrane because of its decreased partitioning in the membrane.

The effect in eukaryotes of losing surface epitope regions spanning residues 171-185 (SEQ ID NO.:4) or 154-219 (SEQ ID NO.:3) was similar to that described above (data not shown; see Yadav, et al., 2004). When H358 cells were transfected with an empty vector (pcDNA3.1) or a vector containing either full length RLIP76 or its deletion mutants lacking aa 171-185 or aa 154-219, membrane association of RLIP76 was significantly reduced in cells transfected with the deletion mutants, as analyzed by Western blots. Hence, the aa 154-219 region is a determinant of the membrane association of RLIP76 and it is independent of whether the protein is expressed in eukaryotes or prokaryotes. Immuno-histochemistry studies using anti-RLIP76 antibodies raised against full-length RLIP76 were performed with live, unfixed H358 wild-type cells and examined by confocal laser microscopy and showed a staining pattern consistent with cell-surface localization. RLIP76 co-localized with another protein, her2/neu, known to have a cell-surface domain. Anti-RLIP76 antibody was detected using a rhodamine red-x-conjugated secondary antibody, and anti-her2/neu antibody using an FITC tagged secondary antibody. Cell-surface epitopes were recognized by both anti-RLIP76 and her2/neu antibodies which co-localized in unfixed cells indicating that RLIP76 had cell-surface epitopes just like her2/neu.

H358 cells constitutively express a wild-type RLIP76. The wild-type was removed by treating H358 cells with si-RNA directed at the region encoding aa 171-185, to silence the expression of wild-type RLIP76, while leaving the expression of 171-185 mutant unaffected. For this, a 23-nucleotide sequence motif comprising AA(N19)TT or NA(N21) (N, any nucleotide) with approximately 50% GC content was searched for. The sequence of sense si-RNA corresponds to N21. The 3′ end of the sense si-RNA was converted to TT to generate a symmetric duplex with respect to the sequence composition of sense and antisense 3′ overhangs. The selected si-RNA sequence was subjected to blast-search (NCBI database) against EST libraries, to ensure that only one gene was targeted. Chemically synthesized si-RNA duplex in the 2′ de-protected and desalted forms were used. A 23-nucleotide long scrambled si-RNA duplex was used as a control. The scrambled si-RNA sequence was not homologous with RLIP76 mRNA in a blast-search against RLIP76. The targeted cDNA sequence was AAGAAAAAGCCAATTCAGGAGCC (SEQ ID NO.:13) corresponding to nucleotides 508 to 528. The corresponding sense si-RNA sequence was GAAAAAGCCAAUUCAGGAGCCdTdT (SEQ ID NO.:14) and the antisense si-RNA sequence was GGCUCCUGAAUUGGCUUUUUCdTdT (SEQ ID NO.:15). The sequences of the scrambled si-RNA in the sense and antisense directions were GUAACUGCAACGAUUUCGAUGdTdT (SEQ ID NO.: 16) and CAUCGAAAUCGUUGCAGUUACdTdT (SEQ ID NO.: 17), respectively.

Transfection of si-RNA duplexes was performed using a kit (Transmessenger Transfection Reagent Kit from Qiagen) and assayed for expression about 24 hours later. Cells (approximately 3×10⁶) were placed into six-well plates and after about 24 hours were incubated for about 3 hours with RLIP76 si-RNA or scrambled si-RNA in an appropriate transfection reagent. Excess si-RNA was washed off with PBS and medium was added. Cell samples were pelleted, solubilized in a lysis buffer (10 mM Tris-HCl, pH 7.4, containing 1.4 mM P-mercaptoethanol, 100 μM EDTA, 50 μM BHT, 100 μM PMSF and 1% polidocanol), sonicated and then incubated for about 4 h in the cold (4° C.). Afterwards, each sample was centrifuged and supernatants (containing both cytosolic proteins and solubilized membrane proteins) collected and analyzed by Western blot analyses according to a method provided by Towbin et al. (Towbin, et al. PNAS 1979; 76:4350-4353) using anti-RLIP76 IgG as well as IgG against the peptide 171-185. Gel bands were quantified by scanning densitometry. Polyclonal antibodies against various deleted epitope regions of RLIP76 were custom made. The peptide antibodies as well as pre-immune serum were purified by DE-52 anion exchange chromatography, followed by protein-A-Sepharose affinity chromatography to obtain pure IgG fractions. Immuno-reactivity and specificity of these peptides using their respective purified IgG were checked by dot blot analyses.

The si-RNA 171-185 effectively silenced wild-type RLIP76 expression in the untransfected, empty-vector-transfected, as well as wild-type RLIP76 transfected cells (data not shown; see Yadav, et al., 2004). Antibodies against the 171-185 peptide failed to detect RLIP76 antigen, while antibodies against full-length RLIP76 recognized the persistent presence of the residual deletion mutant RLIP76. Western blotting against the anti-del 171-185 antibody showed no signal in the RLIP76 deletion mutant transfected cells confirming that expression of wild-type RLIP76 was effectively blocked in these cells. Cell surface expression of RLIP76 in del 171-185 transfected cells with or without pre-treatment with si-RNA directed at aa 171-185 using immunohistochemical analaysis and an anti-del 171-185 antibody showed that cells with control si-RNA had significant cell surface signal which was absent in cells in which RLIP had ben silenced by the si-RNA. RLIP76 is, thus, an integral membrane protein with at least one cell surface domain spanning amino acids 154 to 219.

Accordingly, the present invention provides several surface epitope regions of RLIP that, when altered, blocked or deleted, prevent RLIP from performing its transport function. Compositions of the present invention include the several surface epitope regions as well as use of these surface epitope regions to obtain specific inhibitors of RLIP that are capable of altering, inhibiting or the transport function of RLIP. The inhibitors include si-RNAs, each having a sequence directed against the one or more surface epitope regions as well as phosphorothioate antisense oligonucleotides directed against such surface epitope regions (e.g., GGCTCCTGAATTGGCTTTTTC; SEQ ID NO.:18) and a corresponding silencing RNA sequence to the phosphorothioate antisense oligonucleotides (e.g., AAGAAAAGCCAATTCAGGAGCC; SEQ ID NO.: 19). In addition, inhibitors include antibodies (monoclonal and/or polyclonal) directed against the one or more surface epitope regions, such regions including SEQ ID NO.: 3 and SEQ ID NO.:4. Moreover, the inhibitors identified herein provide compounds for regulation of glucose and insulin. Importantly, the inhibitors are additional targets for identifying important compounds and small molecule from chemical library screenings, wherein the identified compounds and/or small molecules are effective as medicines for glucose and insulin regulation.

Suitable compositions of the present invention also include compounds that interact with RLIP76 through its POB1 binding site and may be identified as described below.

The inventors have recently reported the purification of POB1 and one of its deletion mutant POB1¹⁻⁵¹² (lacking the RLIP76 binding domain) and shown that POB1 regulates the transport function of RLIP76 (further described in Yadav, et al., Biochem. Biophys. Res. Commun. 328:1003-1009; 2005). Both the transport of doxorubicin (DOX) and a predominant GSH-conjugate, dinitrophenyl-S-glutathione (DNP-SG) were found to be inhibited by POB1 in a concentration dependent manner but were not inhibited by POB1^{1-512.} Liposomal delivery of recombinant POB1 to H358 cancer cells caused apoptosis in a concentration dependent manner, whereas POB1¹⁻⁵¹² did not exert this effect. Augmentation of cellular POB1 resulted in increased intracellular DOX accumulation as well as a decrease in the rate of its efflux from cells.

The 2200 bp full length long version cDNA of POB1 (SEQ ID NO.:24) was used as a template for PCR amplification of the POB1 coding sequence. An upstream primer (GGCGGATCCATGGAGGCGGCAGCGGC; SEQ ID NO.:25) and downstream primer (CCGCTCGAGTCACAACACAGTGACCGGAC; SEQ ID NO.:26) were designed to introduce a BamH1 restriction site (underlined) immediately upstream of the initiator codon and Xho I site (underlined) immediately downstream of the stop codon of POB1 open reading frame. PCR amplification was performed under with DNA template, 500 ng; primers, 30 pmol each; dNTPs, 2.5 μM; thermopol buffer, 1×; BSA, 1×; and Vent polymerase, 2.5 U. PCR cycles were: 94° C. for 5 minutes followed by 35 cycles of 94° C. for 30 seconds; 60° C. for 30 seconds and 1 minute at 72° C. and a final extension at 72° C. for 7 minutes. PCR product was purified and digested with BamHI and XhoI restriction enzymes. The cleaved PCR products were ligated into pET30a(+) previously digested with the same restriction enzymes. The ligated products were expressed into DH5α-competent cells and plasmid DNA was purified from an overnight culture of a single colony. Techniques for restriction enzyme digestion, ligation, transformation and other standard molecular biology manipulations are apparent to those of ordinary skill in the art. The sequence of the POB1 was confirmed by DNA sequencing. Following verification of the sequence of POB1, the pET30a(+) plasmid containing the full-length POB1 was used to transform E. coli strain BL21(DE3) and protein was expressed in E. coli BL21(DE3) grown at 37° C. after induction with 0.4 mM IPTG.

Recombinant (rec) POB1 was purified by metal affinity chromatography over a Ni-NTA superflow resin as described in Yadav, et al., Biochem. Biophys. Res. Commun. 328:1003-1009; 2005. Briefly, E. coli BL21(DE3) expressing POB1 were lysed (20 mM Tris-HCl containing 250 mM NaCl; 100 μM PMSF and 5 mM imidazole; pH 7.9) sonicated and incubated for 4 hours at 4° C. with gentle shaking followed by centrifugation. Supernatant was mixed with Ni-NTA Superflow resin pre-equilibrated with the same buffer. The resin was incubated overnight in the cold with gentle shaking and washed with wash buffer (20 mM Tris-HCl, 300 mM NaCl; 20 mM imidazole and 100 μM PMSF; pH 7.9) until OD₂₈₀ was zero. The bound protein from the resin was eluted with elution buffer (20 mM Tris; 500 mM NaCl; 400 mM imidazole and 100 μM PMSF pH 7.9) and dialyzed against a buffer (10 mM Tris-HCl, pH 7.4, 100 μM EDTA, 100 μM PMSF containing 0.025% C₁₂E₉). Protein was estimated by an alternate method described in Minamide and Bamburg, Anal. Biochem. 1990; 190:66-70. Western blot analysis and polyacrylamide gel electrophoresis (PAGE) were performed by methods known in the art.

A deletion mutant of POB1 lacking the RLIP76 binding site, referred to as POB1¹⁻⁵¹², contained the sequence coding for aa 1-512 starting from the first amino acid of the open reading frame (POB1¹⁻⁵¹²; SEQ ID NO.:27 with a nucleotide sequence shown in SEQ IN NO.:28). The mutant was constructed by PCR amplification using full length POB1 as a template. The upstream primer was GGCGGATCCATGGAGGCGGCAGCGGC (SEQ ID NO.:29) containing a BamH1 site (underlined); downstream primer was CCGCTCGAGGGTAACAATCCTGACTTGGTA (SEQ ID NO.:30) containing the XhoI restriction site (underlined). PCR amplification was performed using: DNA template, 500 ng; primers, 30 pmol each; dNTPs, 2.5 μM; thermopol buffer, 1×; BSA, 1×; Vent polymerase, 2.5 U. PCR cycles were: 94° C. for 5 minutes followed by 35 cycles of 94° C. for 30 seconds; 60° C. for 30 seconds; 1 minute at 72° C. and a final extension at 72° C. for 7 minutes. PCR product was purified by using a purification kit and digested with BamHI and XhoI restriction enzymes. The cleaved PCR products were ligated into pET30a(+) previously digested with the same restriction enzymes. The ligated product was expressed into DH5α-competent cells from which plasmid DNA was purified from an overnight culture from a single colony. The sequence of the deletion mutant was confirmed by DNA sequencing. Following verification of the sequence of POB1¹⁻⁵¹², the pET30a(+) plasmid containing the POB1¹⁻⁵¹² was used to transform E. coli strain BL21(DE3) and protein was expressed in E. coli BL21(DE3) grown at 37° C. after induction with 0.4 mM IPTG. POB1¹⁻⁵¹² was purified using similar protocol as described above for full length POB1.

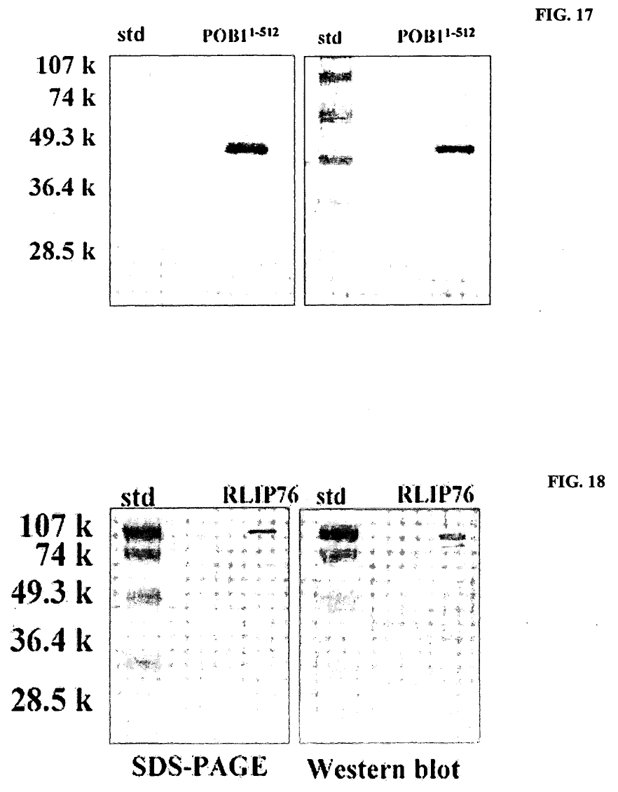

Full length POB1 cDNA as well as a truncated cDNA encoding POB1¹⁻⁵¹² were used to create a His-tagged construct expressed in E. coli. Corresponding proteins were purified and confirmed by SDS-PAGE confirmed; POB1 was 78 kDa and POB1¹⁻⁵¹² was 52 kDa, both recognized by anti-POB1 antibodies in Western blots (FIG. 16 and FIG. 17) as compared with DNP-SG Sepharose-affinity purified rec-RLIP76 (FIG. 18).

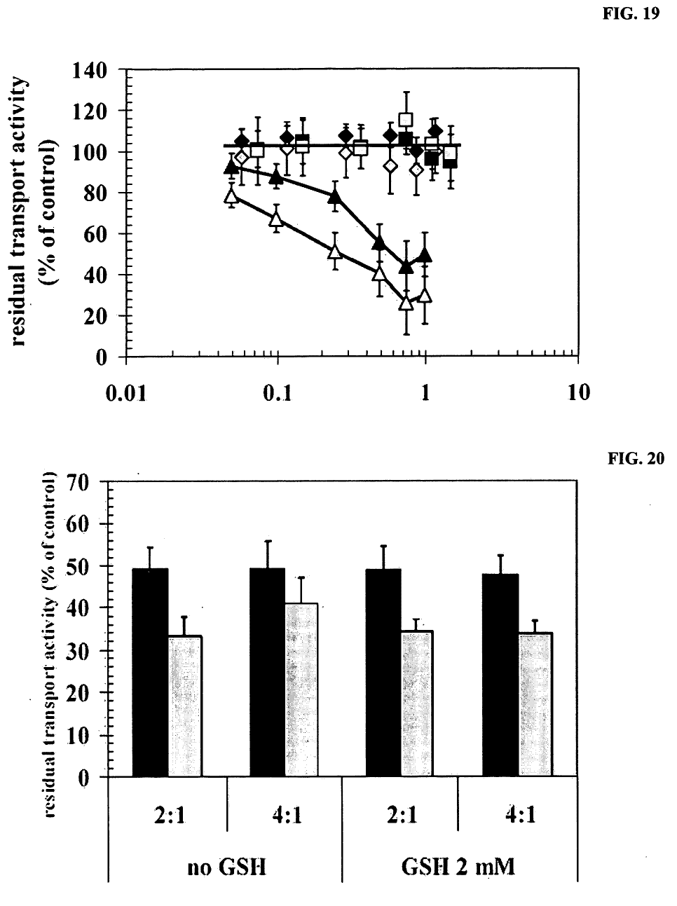

POB1 or POB1⁻⁵¹² (0-80 μg) were reconstituted and ATP-dependent transport of DOX as well as DNP-SG was determined in proteoliposomes containing RLIP76 as shown in FIG. 19. DOX and DNP-SG transport activity were inhibited in a concentration-dependent manner by recombinant POB1, but not by POB1¹⁻⁵¹² or albumin. Maximum inhibition (˜50% for DOX-transport, and 68% for DNP-SG transport) was reached near a 1:1 molar ratio of POB1/RLIP76. Higher molar ratios of POB1/RLIP76 (2:1 or 4:1) did not further inhibit RLIP76 transport activity (FIG. 20). Thus, maximal inhibition of RLIP76 catalyzed transport is achieved at 1:1 stochiometric binding of POB1 to RLIP76. In addition, no effect of 2 mM GSH on POB1 mediated inhibition of RLIP76 was found (FIG. 20). This distinguishes the transport activity of RLIP76 from other transporters such as ABCC1 and indicate that POB1 binding to RLIP76 does not confer the property of GSH-regulated transport.

Prokaryotic expression and purification of bacterially expressed rec-RLIP76 was performed and its purity was established as described previously by the inventors (Awasthi, et al., Biochemistry 2000; 39:9327-9334; Awasthi, et al., Biochemistry 2001; 40:4159-4168). Purified RLIP76 was dialyzed against a reconstitution buffer (10 mM Tris-HCl, pH 7.4, 2 mM MgCl₂, 1 mM EGTA, 100 mM KCl, 40 mM sucrose, 2.8 mM BME, 0.05 mM BHT, 0.025% polidocanol). An aqueous emulsion of soybean asolectin (40 mg/mL) and cholesterol (10 mg/mL) was prepared in the reconstitution buffer by sonication and 0.1 mL of this mixture was added to 0.9 mL aliquot of dialyzed purified rec-RLIP76 containing 20 μg protein. The reaction mixture was sonicated and vesiculation initiated by addition of 200 mg SM-2 Bio-beads pre-equilibrated in the reconstitution buffer without polidocanol. Vesiculation continued for 4 hours, 4° C., followed by removal of the beads by centrifugation. Control vesicles (liposomes) were prepared using an equal amount of crude protein from E. coli not expressing RLIP76. ATP-dependent transport of ¹⁴C-DOX and ³H-DNP-SG in the rec-RLIP76 reconstituted proteoliposomes performed by a rapid filteration technique.

For transport of ¹⁴C-DOX and ³H-DNP-SG and its inhibition by POB1 and POB1¹⁻⁵¹², a fixed amount of purified rec-RLIP76 (20 μg) was reconstituted in a proteoliposome preparation along with varying amounts (0-80 μg) of either POB1 or its deletion mutant (POB1¹⁻⁵¹²). Transport of ¹⁴C-DOX and ³H-DNP-SG was then measured. In one control, POB1 proteins were excluded while in other control equivalent amount of BSA were reconstituted in proteoliposomes.

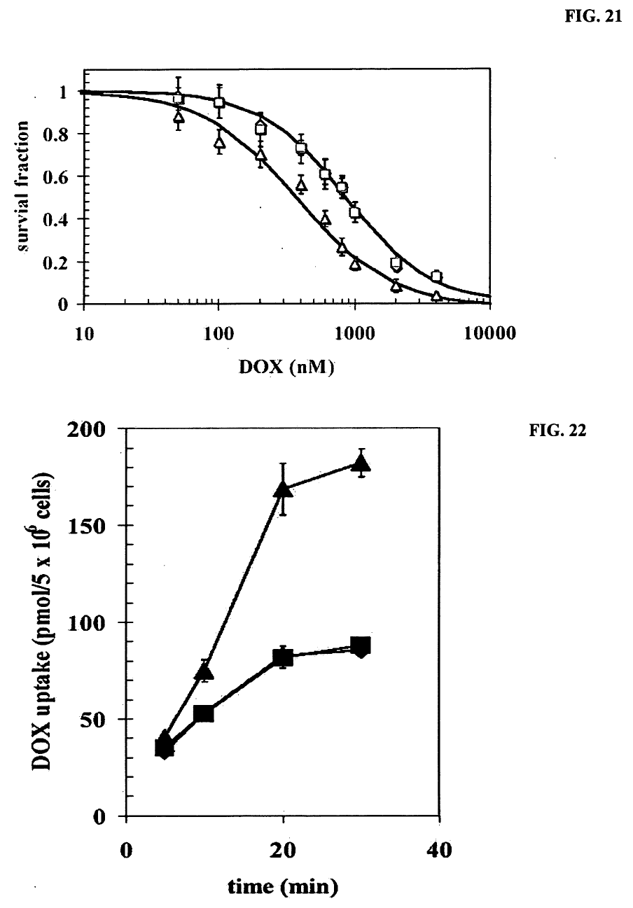

The effect of POB1 on DOX-sensitivity was determined using H358 cells were treated with control liposomes or liposomes containing recombinant POB1 or POB1¹⁻⁵¹² (final concentration 40 μg/ml; an overloaded amount of protein). DOX was added and IC₅₀ measured via MTT assay assessed about 96 hours later as described by the inventors in Awasthi et al., Int. J Cancer 1996; 68:333-339. Treatment with POB1-containing liposomes increased the sensitivity of H358 cells to DOX by about 3-fold, whereas POB1¹⁻⁵¹² had no significant effect on DOX-sensitivity (FIG. 21). Since POB1¹⁻⁵¹², which does not inhibit DOX transport, has no effect on the sensitivity of the cells to DOX, these results indicate that inhibition of RLIP76-mediated DOX-efflux by POB1 sensitizes these cells to DOX through increased intracellular accumulation of the drug. Lack of any effect of BSA on transport of DOX or the sensitivity of cells to DOX rules out nonspecific binding effect of protein(s) on these parameters.

Cell density during the log phase was determined by counting trypan blue excluding cells in a hemocytometer, and 20, 000 cells were plated into each well of 96 well flat bottomed microtiter plates. After 24 hours of incubation, the cells were treated with control liposomes or liposomes containing recombinant full length POB1 or POB1¹⁻⁵¹² (final concentration 40 μg/mL). DOX was added and IC₅₀ was measured by performing MTT assay 96 h later using methods known in the art. Eight replicate wells were used for each point in each of three separate measurement of IC₅₀.

H358 cells were treated with equal amounts of proteoliposomes reconstituted with either recombinant full length POB1 or POB1¹⁻⁵¹² protein. Final concentration of recombinant POB1 or POB1¹⁻⁵¹² in proteoliposome was 40 μg/mL. Results of apoptosis measurement showed that the full length POB1 caused apoptosis in cells while the POB1¹⁻⁵¹² which did not inhibit transport properties of RLIP76, did not have any significant apoptotic effect (data not shown). These results show that inhibition of RLIP76 results in increased accumulation of glutathione-conjugates of endogenously generated toxicants such as 4-HNE, and culminates in apoptosis. Here, H358 cells were grown on the cover slips. The cells were treated with equal amounts of liposomes reconstituted with 40 μg/mL (final concentration) recombinant full length POB1 and POB1¹⁻⁵¹² protein. After 24 h incubation, the medium was removed, and cells were washed with PBS four times. A TUNEL assay was performed using methods known in the art. Fluorescence micrographs were taken using a laser scanning fluorescence microscope at 400× magnification.

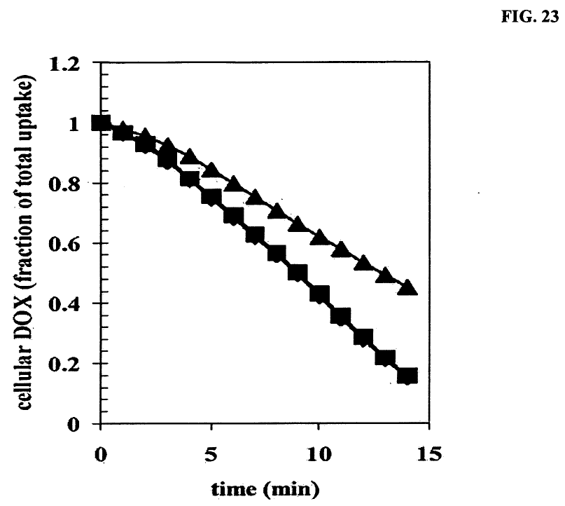

Accumulation and efflux with control, purified rec-POB1 or purified rec-POB1¹⁻⁵¹² liposomes (40 μg/mL final concentration) treated intact H358 cells were performed. For uptake studies, cellular DOX accumulation was quantified at varying times after addition of drug to the extracellular medium (FIG. 22). The total DOX accumulation was markedly increased in rec-POB1 liposomes treated cells, and consistent with the previous transport data. For measuring efflux of DOX, cells were loaded with liposomes and ¹⁴C-DOX by incubating for 60 minutes, followed by rapid dilution in drug-free medium, and taking sequential aliquots of external medium for radioactivity counting. Cell-associated drug was calculated by back-addition using methods known in the art, and plotted with respect to time. The rate of loss of DOX from cells due to efflux was significantly lower for rec-POB1 liposomes treated cells as compared with the control cells (FIG. 23); total cellular DOX accumulation was increased more than 2-fold as compared with cells treated with a control. Here, H358 cells were harvested, washed, and aliquots containing 5×10⁶ cells (in triplicate, for each time point) were inoculated into fresh medium. After overnight incubation, the cells were pelleted and re-suspended in 80 μL medium containing 4 μg either control, POB1 or POB1¹⁻⁵¹² liposomes, and incubated at 37° C. for 24 hours. After 24 hours incubation, 20 μL of 14-[¹⁴C]-DOX (final: 3.6 μM; specific activity: 8.5×10⁴ cpm/nmol) was then added to the medium and incubated for 5, 10, 20, and 30 minutes at 37° C. Drug uptake was stopped by rapid cooling on ice. Cells were centrifuged for 5 minutes at 4° C. and medium was completely decanted. Radioactivity was determined in the cell pellet after washing twice with ice-cold PBS.

For ¹⁴C-DOX efflux studies, H358 cells were harvested washed with PBS and aliquots containing 5×10⁶ cells (in triplicate) were inoculated into fresh medium. After overnight incubation, the cells were pelleted and resuspended in 80 μL medium containing 4 μg either control, POB1 or POB1¹⁻⁵¹² liposomes, and incubated at 37° C. for 24 hours. After 24 hours incubation, 20 μL of 14-[¹⁴C]-DOX (final 3.6 μM, specific activity 8.5×10⁴cpm/nmol) was then added to the medium and incubated for 60 minutes at 37° C. Cells were centrifuged for 5 minutes, after which the supernatant was removed completely and the cell pellet washed twice. The pellet was immediately resuspended in 1 mL of PBS. 50 μL aliquots (clear supernatant) were removed every minute for radioactivity counting for 15 minutes. The back-added curves of cellular residual VRL versus time were constructed as described previously.

Thus, regulation of RLIP76 transport activity by POB1 may serve not only an important role in signaling but may also be relevant to the mechanisms of drug resistance

While particular embodiments of the invention and method steps of the invention have been described herein, additional alternatives not specifically disclosed but known in the art are intended to fall within the scope of the invention. Thus, it is understood that other embodiments and applications of the present invention will be apparent to those skilled in the art without departing from the scope and spirit of the appended claims and drawings.

What is claimed is:

1. A composition comprising:
a region recognizing a ralA binding protein 1, wherein the region further comprises SEQ ID NO.:28 and modified variants thereof, including deletions of 1 to 15 nucleotides at its C-terminus and N-terminus.

2. The composition of claim 1, wherein the composition is used to identify antibodies that effect transport activity and membrane association of the ralA binding protein 1.

3. The composition of claim 1, wherein the composition is used to identify compounds that recognize the ralA binding protein 1.

4. The composition of claim 3, wherein the compounds directly effect transport activity and membrane association of ralA binding protein 1.

5. The composition of claim 3, wherein the compounds are medicines used for regulation of glucose in subjects in need thereof.

6. The composition of claim 3, wherein the compounds are medicines used for regulation of insulin in subjects in need thereof.

7. The composition of claim 1, wherein the composition codes for SEQ ID NO.:27 and modified variants, thereof.

8. The composition of claim 7, wherein SEQ ID NO.:27 and modified variants thereof are used to generate antibodies to the ralA binding protein 1.

9. The composition of claim 7, wherein SEQ ID NO.:27 and modified variants thereof are used for screening chemical libraries for compounds that recognize the ralA binding protein 1.

10. The composition of claim 9, wherein the compounds are medicines used for regulation of glucose in subjects in need thereof.

11. The composition of claim 9, wherein the compounds are medicines used for regulation of insulin in subjects in need thereof.

12. The composition of claim 1, wherein the composition is used to create si-RNA sequences to silence expression of wild-type ralA binding protein 1.

13. A method comprising the steps of:
providing to a subject in need thereof a derived composition provided by the composition of claim 1, wherein the derived composition is an antibody, si-RNA, small molecule and combinations thereof that directly effects transport activity of a ralA binding protein 1.

14. The method of claim 13, wherein the derived composition is used alone or in combination as medicine for regulation of glucose.

15. The method of claim 13, wherein the derived composition is used alone or in combination as medicine for regulation of insulin.

16. The method of claim 12, wherein the derived composition is identified by screening chemical libraries for compounds that recognize the ralA binding protein 1.

17. A method comprising the steps of:
providing to a subject in need thereof a derived composition provided by SEQ ID NO.:22, wherein the derived composition is an antibody, si-RNA, small molecule and combinations thereof that directly effects transport activity of a ralA binding protein 1 and regulates glucose.

18. The method of claim 17, wherein the derived composition is used alone or in combination as medicine for regulation of insulin.

19. The method of claim 17, wherein the derived composition is identified by screening chemical libraries for compounds that recognize the ralA binding protein 1.

20. The method of claim 17, wherein the derived composition is obtained by raising antibodies to ralA binding protein 1.

21. A method comprising the steps of:
providing to a subject in need thereof a derived composition provided by SEQ ID NO.:3, wherein the derived composition is an antibody, si-RNA, small molecule and combinations thereof that directly effects transport activity of a ralA binding protein 1 and regulates glucose.

22. The method of claim 21, wherein the derived composition is used alone or in combination as medicine for regulation of insulin.

23. The method of claim 21, wherein the derived composition is identified by screening chemical libraries for compounds that recognize SEQ ID NO.:3.

24. The method of claim 21, wherein the derived composition is obtained by raising antibodies to SEQ ID NO.:3.
